(19) United States
(12) Patent Application Publication
(10) Pub. No.: US 2014/0066449 A1
(43) Pub. Date: Mar. 6, 2014

(54) DIPYRIDINIUM DERIVATIVES

(71) Applicant: John Charles Marshall Stewart, Rudgwick (GB)

(72) Inventor: John Charles Marshall Stewart, Rudgwick (GB)

(73) Assignee: SABREPHARM LIMITED, Cobham, Surrey (GB)

(21) Appl. No.: 14/002,530

(22) PCT Filed: Mar. 2, 2012
(86) PCT No.: PCT/GB2012/000212

§ 371 Date: Nov. 11, 2013

(30) Foreign Application Priority Data
Mar. 2, 2011 (GB) 1103578.9

(51) Int. Cl. A61K 31/4985; A61K 45/06; C07D 471/14

(52) U.S. Cl. CPC A61K 31/4985; C07D 471/14; A61K 45/06
USPC 514/250; 544/344

(57) ABSTRACT

Compounds of Formula (I): α-x-β (I) and pharmaceutically acceptable salts and solvates thereof, wherein α, x, and β have the meanings as indicated in the specification, are useful for treating a disease or disorder characterised by pathologically proliferating cells, particularly cancer. Pharmaceutical compositions that contain the compounds and processes for preparing the compounds are also described.

This invention relates to dipyridinium derivatives and their use as pharmaceuticals, in particular for the treatment of a disease or disorder characterised by pathologically proliferating cells, particularly cancer.

Cancer is a disordered and uncontrolled growth of tissue, which, if untreated will eventually cause death by destroying the function of the organ in which it arises, and other vital organs to which it may spread.

The principle treatments are surgery, radiotherapy and systemic therapies, including chemotherapy. Of these, surgery and radiotherapy can cure about 50% of cases. However cancers have often spread (metastasised) from their bodily origin to distant parts, when patients first seek medical attention, thus limiting what can be achieved by surgery or radiotherapy alone. At that stage of the disease systemic therapy is a necessary part of management.

Current drug treatments for cancer typically use either highly toxic compounds, which are only partially selective to the tumour and hence cause unpleasant side effects such as nausea, vomiting or hair loss; or compounds which inhibit or interrupt the biological processes supporting tumour growth. As these biological processes adapt to chemical assault over time and result in ‘drug resistance’, treatments become ineffective. In either case, toxicity can significantly limit the use of the drug, and the development of resistance can limit the benefit of therapy. The result is that while certain cancers can be treatable, the overall improvement in longevity for many patients is still very unsatisfactory and the quality of life unacceptable.

Systemic anti-cancer agents kill, or inhibit the growth of malignant cells by many different mechanisms. Examples include inhibiting DNA synthesis, blocking activity of hormones, and inhibiting over-expressed cell surface-receptors with antibodies, The [relative] selectivity of these agents for cancer over normal tissue depends upon the higher growth rate of the cancer compared to normal tissue, cancer-specific features like a unique cell surface antigen, and careful drug-administration regimens in clinical practice: However it is still difficult to deliver an anti-cancer agent to tumours precisely and avoid unwanted clinical side effects.

Targeted anti-cancer agents have been developed in an effort to improve selectivity. These agents inhibit overactive enzymes associated with rapid or uncontrolled growth of the cancer. Drugs of this class can be highly specific and well tolerated, but the number of useful agents so far to emerge from this class has fallen well below the expectations once held out for them (Román Pérez-Soler, HER1/EGFR Targeting: Refining the Strategy, Oncologist, February 2004; 9: 58-67) despite notable early successes for Chronic Myeloid leukaemia, GIST (a rare gastro intestinal stromal tumour), and renal cancer (Wandena S. Siegel-Lakhai, Jos H. Beijnen, Jan H. M. Schellens, Current Knowledge and Future Directions of the Selective Epidermal Growth Factor Receptor Inhibitors Erlotinib (Tarceva®) and Gefitinib (Iresse®), Oncologist, September 2005; 10: 579-589).

Whilst not bound by theory, in part this is considered to be because all inhibitory therapies [including targeted therapies] have inherent defects which are very difficult to overcome. For example, the agent can often block one enzyme so specifically that alternative redundant or mutated enzymes soon emerge and circumvent the inhibition, negating the growth-inhibitory effect of the compound in the process. (Eric K. Rowinsky, Signal Events: Cell Signal Transduction and Its Inhibition in Cancer Oncologist, December 2003; 8: 5-17). The complexity of changes that can occur in cancer biology is revealed in metastasised tumours with different mutations at the different sites of metastases. Increasingly too many different isoforms of a target enzyme are being found in different populations. These isoforms have slight structural differences, which can often profoundly alter the potency of the inhibitor and limit its potential for widespread use.

A further problem for inhibitory therapies is the heterogeneity of tumours. Most tumours, and especially those which are advanced, contain large populations of cells (even 20% to 30% of the total) which do not express the target enzyme, and thus escape inhibition altogether, Unchallenged, these cells grow and become the dominant population when those with a target are selectively inhibited. By this mechanism the emergence of resistant clones are actively encouraged. (Klein S, McCormick F, Levitzki A Killing time for cancer cells. Nat Rev Cancer. 2005 July; 5(7):573-80). Eventually these clones lead to clinical-level disease (clinical progression).

Together the combined influences of enzyme redundancy, mutation isoforms, and the cellular heterogeneity of tumours account for much of the resistance and refractoriness of cancers seen in present-day clinical practice (Kamb A, Wee S, Lengauer C, Why is cancer drug discovery so difficult?, Nat Rev Drug Discov. 2007 February; 6(2):115-20; Hanash S, Integrated global profiling of cancer, Nat Rev Cancer. 2004 August; 4(8):638-44).

Oncologists minimise these problems by using multi drug regimes that act at different points in a cell's metabolism—thus limiting dependence on one metabolic process—and by using different combinations of drugs in a sequence of regimens. These measures improve responses, but even so, resistance and refractoriness invariably emerge for multi drug regimes too.

In one aspect, the invention provides a compound of Formula I:

α-x-β (I)

or a pharmaceutically salt or solvate thereof, wherein
α is polyethylene glycolyl or H;
X is a linker group wherein the linker group is selected from —O—, —C(O)NR¹⁰—, —NR¹⁰C(O)NR¹¹—, —NR¹⁰C(O)O—, —NR¹⁰—, —C(O)O—, —S—, —(SO₂)NR¹⁰—, —(SO₂)O—, —NR¹⁰(SO₂)O—, —NR¹⁰(SO₂)NR¹¹—, —NR¹⁰C(O)NR¹¹(CH₂)ₙNR^{10a}C(O)O—, —OC(O)NR¹⁰(CH₂)ₙNR^{10a}C(O)NR^{11a}—, —NR¹⁰C(O)NR¹¹(CH₂)ₙNR^{10a}C(O)NR^{11a}—, and —OC(O)NR¹⁰(CH₂)ₙNR^{10a}C(O)O—, wherein each (CH₂) is optionally substituted by one or more halogen atoms, hydroxyl, C₁-C₄ alkoxy, C(O)NH₂, C(O)NHC₁-C₆ alkyl or C(O)N(C₁-C₆ alkyl)₂;
R¹⁰, R^{10a}, R¹¹ and R^{11a} are independently selected from H, C₁-C₈ alkyl; C₃-C₈ cycloalkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; wherein the alkyl groups are optionally substituted by one or more halogen atoms, hydroxyl, C₁-C₄ alkoxy, C(O)NH₂, C(O)NHC₁-C₆ alkyl or C(O)N(C₁-C₆ alkyl)₂;
n is 1, 2, 3, 4, 5 or 6;
β is a dipyridinium salt wherein the dipyridinium salt is of Formula 2

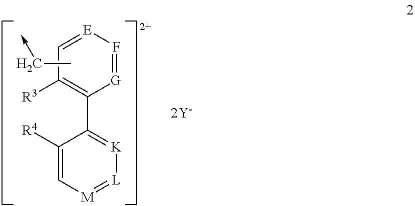

wherein E, F, G, K, L and M are each independently selected from CR¹ and NR² with the proviso that E, F or G is NR² and K, L or M is NR² and only one of E, F and G is NR² and only one of K, L and M is NR²; any pyridyl carbon atom may be the site of substitution of the methylene group bonded to the X linker;
R¹ and R² are independently selected from H and C₁₋₃alkyl; or
wherein G and K are both NR², the two R² groups may be joined to form a CR¹²R¹³CR¹⁴R¹⁵ bridge;
R³ and R⁴ are independently selected from H and C₁₋₃alkyl; or
R³ and R⁴ are joined to form a CR¹⁶CR¹⁷ bridge
R¹², R¹³, R¹⁴ and R¹⁵ are independently selected from H, C₁-C₈ alkyl; C₃-C₈ cycloalkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; wherein the alkyl groups are optionally substituted by one or more halogen atoms, hydroxyl, C₁-C₄ alkoxy, C(O)NH₂, C(O)NHC₁-C₆ alkyl or C(O)N(C₁-C₆ alkyl)₂;
R¹⁶ and R¹⁷ are independently selected from H, C₁-C₈ alkyl; C₃-C₈ cycloalkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; wherein the alkyl groups are optionally substituted by one or more halogen atoms, hydroxyl, C₁-C₄ alkoxy, C(O)NH₂, C(O)NHC₁-C₆ alkyl or C(O)N(C₁-C₆ alkyl)₂;
Y⁻ is independently a pharmaceutically acceptable counteranion of an inorganic or organic acid; and
the arrow head denotes the point of attachment to X.

In one embodiment of the invention as defined anywhere above α is H

In an alternative embodiment of the invention as defined anywhere above α is polyethylene glycolyl.

In a further embodiment of the invention as defined anywhere above α is polyethylene glycolyl of molecular weight 100 to 20,000 daltons.

In a further embodiment of the invention as defined anywhere above α is polyethylene glycolyl of molecular weight 1000 to 10,000 daltons.

In a further embodiment of the invention as defined anywhere above α is methoxy polyethylene glycolyl.

In one embodiment of the invention as defined anywhere above, R¹ is H

In another embodiment of the invention as defined anywhere above, X is selected from —O—, —C(O)NR¹⁰—, —NR¹⁰C(O)NR¹¹—, —NR¹⁰C(O)O—, —NR¹⁰—, —C(O)O—, —NR¹⁰C(O)NR¹¹(CH₂)ₙNR^{10a}C(O)O—, —OC(O)NR¹⁰(CH₂)ₙNR^{10a}C(O)NR^{11a}—, —NR¹⁰C(O)NR¹¹(CH₂)ₙNR^{10a}C(O)NR^{11a}—, and —OC(O)NR¹⁰(CH₂)ₙNR^{10a}C(O)O—.

In another embodiment of the invention as defined anywhere above, R¹⁰, R^{10a}, R¹¹ and R^{11a} are H.

In another embodiment of the invention as defined anywhere above G and K are both NR², and the two R² groups are joined to form a C₂H₄ alkylene bridge

In a further embodiment of the invention as defined anywhere above, R³ and R⁴ are H

In an alternative further embodiment the invention as defined anywhere above R³ and R⁴ are joined to form a C₂H₂ alkenyl bridge

In another embodiment of the invention as defined anywhere above, R¹², R¹³, R¹⁴ and R¹⁵ are H.

In another embodiment of the invention as defined anywhere above, R¹⁶ and R¹⁷ are H.

In another embodiment of the invention as defined anywhere above 13 is a dipyridinium salt of Formula 2a

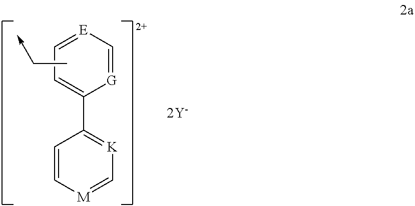

wherein E, G, K, M are each independently selected from CR¹ and NR² with the proviso that E or G is NR² and K or M is NR² and E and G are not both NR² and K and M are not both NR² wherein R¹, R² and r are as defined anywhere hereinabove.

In another embodiment of the invention as defined anywhere above β is a dipyridinium salt of Formula 2b

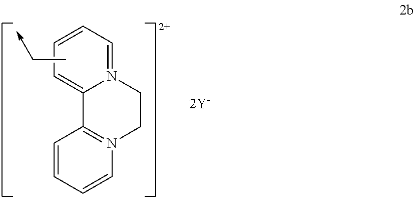

wherein Y⁻ is as defined anywhere hereinabove.

In another embodiment of the invention as defined anywhere above β is a dipyridinium salt of Formula 2c

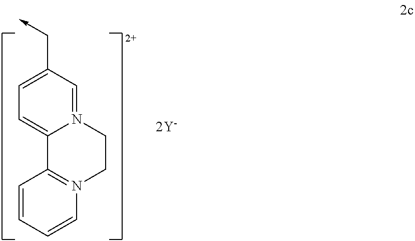

wherein Y⁻ is as defined anywhere hereinabove.

In another embodiment of the invention as defined anywhere above β is a dipyridinium salt of Formula 2d

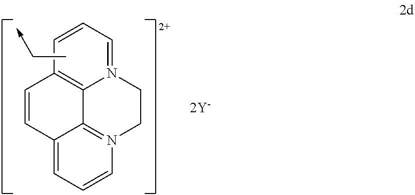

wherein Y⁻ is as defined anywhere hereinabove.

In another embodiment of the invention as defined anywhere above the counteranion Y⁻ is independently selected from acetate, aspartate, benzoate, besylate, bromide/hydrobromide, bicarbonate/carbonate, bisulfate/sulfate, cam phorsulformate, chloride/hydrochloride, chlortheophyllonate, citrate, ethandisulfonate, fumarate, gluceptate, gluconate, glucuronate, hippurate hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate, trifluoroacetate, and trifluoromethylsulphonate.

The compounds of formula I are charge balanced and a skilled person will appreciate that if the counteranion is divalent, 2Y⁻ represents a single counteranion.

A skilled person will further appreciate that 2Y⁻ may represent two identical or two different monovalent counteranions.

In a further embodiment of the invention as defined anywhere above, Y⁻ is trifluoromethylsulphonate

In a yet further embodiment, the invention provides a compound of Formula Ia

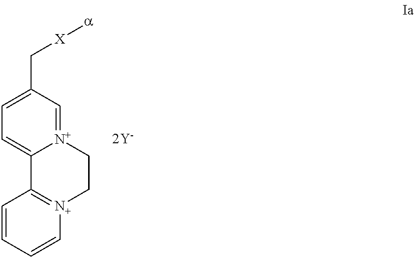

or a pharmaceutically acceptable salt or solvate thereof wherein α, X and Y are as defined anywhere hereinabove in respect of a compound of Formula I.

The present invention also provides compounds wherein the polyethylene glycolyl group is multifunctional and bonded to a plurality of dipyridinium moieties of formula X-β wherein X and β are as defined anywhere above in respect of a compound of Formula I and each dipyridinium moiety of formula X-β may be the same or different.

In one embodiment, the present invention provides a compound of formula Ih

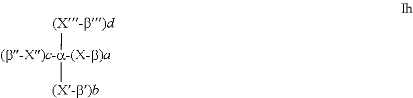

or a pharmaceutically acceptable salt or solvate thereof wherein α is polyethylene glycolyl or H, wherein when α is H or a monofunctional polyethylene glycolyl, α is 1, b is 0, c is 0 and d is 0; X and 13 are as defined anywhere above in respect of a compound of Formula I and X′ and β′, X″ and β″, X′″ and β′″ are independently as defined anywhere above in respect of X and β respectively, and a, b, c and d are independently 0 or 1 wherein at least one of a, b, c and d is 1.

In another embodiment, the present invention provides a compound of formula Ig

β′-x′-α-x-β Ig

or a pharmaceutically acceptable salt or solvate thereof wherein α is a bifunctional polyethylene glycolyl group, X and β are as defined anywhere above in respect of a compound of Formula I and X′ and β′ are independently as defined anywhere above in respect of X and β respectively.

In a further embodiment, the invention provides a compound of Formula Ij

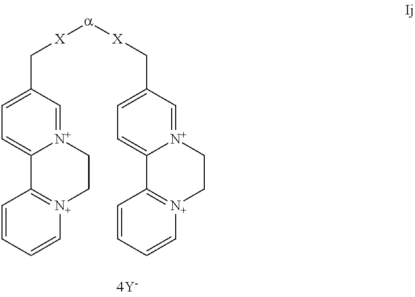

or a pharmaceutically acceptable salt or solvate thereof wherein α, X and Y are as defined anywhere above in respect of a compound of Formula I.

In one embodiment, X, X′, X″ and X′″ are identical when present.

In one embodiment, β, β′, β″ and β are identical when present

In another embodiment, individual compounds according to the invention are those listed in the Examples section below.

In another embodiment of the invention, there is provided a compound according to the invention which is selected from Examples 1, 2, 3, 4, 4.1 and 4.2 or a pharmaceutically acceptable salt or solvate thereof.

In another embodiment of the invention, there is provided a compound according to the invention which is selected from:

### 3-(hydroxymethyl)-6,7-dihydrodipyrido[1,2-a:2′,1′-c]pyrazine-5,8-diium bistrifluoromethanesulfonate

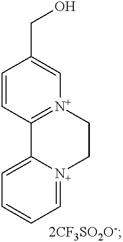 [SMILES: OCc1ccc2[n+](c1)CC[n+]1ccccc1-2]

### 3-((2-(2-methoxy-ethoxy)(ethoxy)ₙ)methyl)-6,7-dihydrodipyrido[1,2-a:2′,1′-c]pyrazine-5,8-diium bistrifluoromethanesulfonate

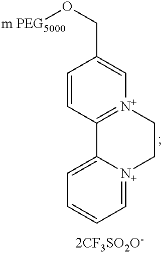

### 3-(((2-(2-methoxyethoxy)(ethoxy)ₙ)ethyl) hexane-1,6-diyldicarbamate)methyl)-6,7-dihydrodipyrido[1,2-a:2′,1′-c]pyrazine-5,8-diium bistrifluoromethanesulfonate

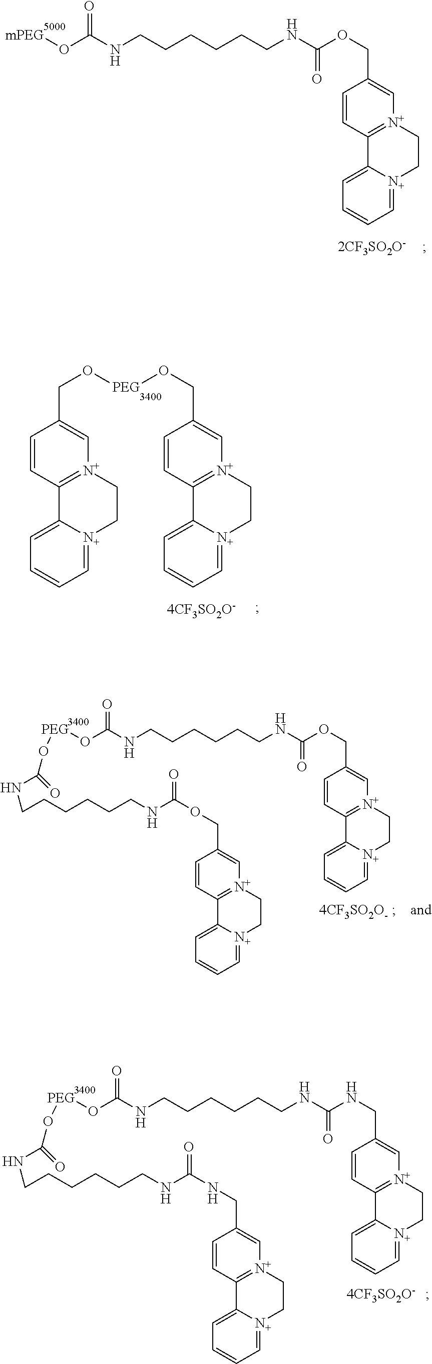

or a pharmaceutically acceptable salt or solvate thereof.

In the embodiments mentioned herein, where only certain variables are defined, it is intended that the remainder of the variables are as defined in any embodiment herein. Thus, the invention provides for the combination of limited or optional definitions of variables.

The following terms as used herein are intended to have the following meanings:

Polyethylene glycolyl as used herein means a covalently bonded polyethylene glycol group. Polyethylene glycol (PEG) is a polyether compound which may also known as polyethylene oxide (PEO) or polyoxyethylene (POE). PEG, PEO, or POE refers to an oligomer or polymer of ethylene oxide which has the following structure:

H—(O—CH₂—CH₂)ₙ—OH.

The three names are chemically synonymous and in this context, the term “polyethlene glycolyl” is not limited by molecular weight. PEGs are prepared by polymerization of ethylene oxide and are commercially available over a wide range of molecular weights from 300 g/mol to 10,000,000 g/mol. Different forms of PEG are also available dependent on the initiator used for the polymerization process, the most common of which is a monofunctional methyl ether PEG (methoxypoly(ethylene glycol)), abbreviated mPEG. A linear PEG has an α-end and an ω-end. If one end is carrying a methoxy group it is unreactive at this end and cannot be used for any further chemical modification. The other end still carrying a reactive group can form a conjugate. The PEG is then called monofunctional and can be used to PEGylate. The α-end and the ω-end can carry the same or different functional group, and are classified as homo- or heterobifunctional PEGs respectively. If both ends are carrying a reactive group, the PEG is bifunctional and can be used as linker between two compounds. In this context, the term “polyethlene glycolyl” includes such mono and bifunctionalised PEG.

PEGs are also available with different geometries: Branched PEGs have typically 3 to 10 PEG chains emanating from a central core group; Star PEGs have typically 10-100 PEG chains emanating from a central core group; Comb PEGs have multiple PEG chains typically grafted to a polymer backbone. In this context, the term “polyethlene glycolyl” includes such geometric isomers. Such non-linear PEGs may contain a plurality of functional groups. In particular, a non-linear PEG may be mono, bi, tri or tetra-functional.

PEGs are typically quoted with the number to indicate their average molecular weights, e.g., a PEG with n=9 would have an average molecular weight of approximately 400 daltons and would be labeled PEG 400. Most PEGs include molecules with a distribution of molecular weights; i.e., they are polydisperse. The size distribution can be characterized statistically by its weight average molecular weight (Mw) and its number average molecular weight (Mn), the ratio of which is called the polydispersity index (Mw/Mn). Mw and Mn can be measured by mass spectrometry.

“PEGylation” as used herein is the act of covalently coupling the PEG structure Q to the linker group X (which is then referred to as PEGylated).

“Optionally substituted” as used herein means the group referred to can be unsubstituted, or substituted at one or two or three positions by any one or any combination of the radicals listed thereafter.

“Halo” or “halogen” as used herein means fluorine, chlorine, bromine or iodine.

“C₁-C₃ alkyl”, “C₁-C₆ alkyl”, “C₁-C₈ alkyl” and the like, as used herein, denotes a straight chain or branched alkyl group that contains one to three, six or eight (or the relevant number) carbon atoms and which may be substituted as defined.

“Aryl”, as used herein, represents an aromatic carbocyclic ring system having 6 to 15 carbon atoms. It can be monocyclic, bicyclic or tricyclic, and may be optionally substituted as defined. Examples of C₆-C₁₅-aryl groups include but are not limited to phenyl, phenylene, benzenetriyl, indanyl, naphthyl, naphthylene, naphthalenetriyl and anthracenyl.

“Heterocyclyl” or “heterocyclic” refers to a 4- to 14-membered heterocyclic ring system containing at least one ring heteroatom selected from the group consisting of nitrogen, oxygen and sulphur, which may be saturated, partially saturated or aromatic (i.e. heteroaryl). Examples of 4- to 14-membered heterocyclic groups include but are not limited to furan, azetidine, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, pyrrolidinone, pyridinone, morpholine, triazine, oxazine, tetrahydrofuran, tetrahydrothiophene, tetrahydrothiopyran, tetrahydropyran, 1,4-dioxane, 1,4-oxathiane, indazole, quinoline, quinazoline, quinoxaline, indole, indoline, thiazole, thiophene, isoquinoline, isoindole, isoindoline, benzothiophene, benzoxazole, benzisoxazole, benzothiazole, benzisothiazole, benzofuran, dihydrobenzofuran, dihydroisobenzofuran, benzodioxole, benzimidazole, benzotriazole, pyrazolopyridine, pyrazolopyrimidine, imidazopyridine, purine, naphthyridine or tetrahydronaphthyridine. “Heterocyclyl” or “heterocyclic” also includes bridged heterocyclic groups such as 3-hydroxy-8-aza-bicyclo[3.2.1]oct-8-yl and fused ring systems such as pyridopyrimidine. The 4- to 14-membered heterocyclic group can be unsubstituted or substituted.

“Heterocyclyl” includes heteroaryl and heterocycloalkyl groups.

“Heteroaryl” is an aromatic ring system containing from 5 to 15 ring atoms one or more of which are heteroatoms selected from O, N or S. Preferably there are one or two heteroatoms. Heteroaryl (heterocyclic aryl) represents, for example: pyridyl, indolyl, isoindolyl, indazolyl, quinoxalinyl, quinazolinyl, quinolinyl, isoquinolinyl, naphthryridinyl, pyridopyrimidinyl, benzothienyl, benzofuranyl, benzopyranyl, benzothiopyranyl, benzotriazolyl, pyrazolopyridinyl, furanyl, pyrrolyl, thiazolyl, oxazolyl, isoxazolyl, triazolyl, tetrazolyl, pyrazolyl, imidazolyl, thienyl. The heteroaryl group can be substituted or unsubstituted.

“C₃-C₁₀-cycloalkyl” denotes a fully saturated carbocyclic ring having 3 to 10 ring carbon atoms, for example a monocyclic group such as a cyclopropyl, cyclobutyl, cyclopentyl or cyclohexyl, cycloheptyl, cyclooctyl, cyclononyl or cyclodecyl, or a bicyclic group such as bicycloheptyl or bicyclooctyl. Different numbers of carbon atoms may be specified, with the definition being amended accordingly. The cycloalkyl group can be substituted or unsubstituted.

“C₅-C₁₀-cycloalkenyl” denotes a partially saturated carbocyclic ring having 5 to 10 ring carbon atoms, for example a monocyclic group such as a cyclopentenyl or cyclohexenyl, cycloheptenyl, cyclooctenyl or cyclononenyl, or a bicyclic group such as bicycloheptenyl or bicyclooctenyl. The ring or ring system may contain more than one carbon-carbon double bond. Different numbers of carbon atoms may be specified, with the definition being amended accordingly. The cycloalkenyl group can be substituted or unsubstituted.

“C₁-C₈-haloalkyl” as used herein denotes C₁-C₈-alkyl as hereinbefore defined substituted by one or more halogen atoms, preferably one, two or three halogen atoms. Different numbers of carbon atoms may be specified, with the definition being amended accordingly.

“C₁-C₈-alkylamino” as used herein denote amino substituted by one or two C₁-C₈-alkyl groups as hereinbefore defined, which may be the same or different. Different numbers of carbon atoms may be specified, with the definition being amended accordingly.

“C₁-C₈-alkoxy” as used herein denotes straight chain or branched alkoxy that contains 1 to 8 carbon atoms. Different numbers of carbon atoms may be specified, with the definition being amended accordingly.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadec, anoate, oleate, oxalate, palmitate, pamoate, phosphate/hydrogen phosphate/dihydrogen phosphate, polygalacturonate, propionate, stearate, succinate, sulfosalicylate, tartrate, tosylate, trifluoroacetate and trifluoromethylsulfonate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoromethylsulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic on exchange resins, and the like. Certain organic amines include isopropylamine, benzathine, cholinate, diethanolamine, diethylamine, lysine, meglumine, piperazine and tromethamine.

Furthermore, the compounds of the present invention, including their salts, can also be obtained in the form of their hydrates, or include other solvents used for their crystallization.

Compounds of the invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included. Tautomers are one of two or more structural isomers that exist in equilibrium and are readily converted from one isomeric form to another.

Examples of tautomers include but are not limited to those compounds defined in the claims.

Any asymmetric atom (e.g., carbon or the like) of the compound(s) of the present invention can be present in racemic or enantiomerically enriched, for example the (R)—, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

When both a basic group and an acid group are present in the same molecule, the compounds of the present invention may also form internal salts, e.g., zwitterionic molecules.

Whilst not bound by theory, the present invention provides compounds according to the invention wherein α is polyethylene glycolyl that are prodrugs which convert in vivo to active dipyridinium compounds. A pro-drug is an active or inactive compound that is modified chemically through in vivo physiological action, such as hydrolysis, metabolism and the like, into a compound of the invention following administration of the prodrug to a subject. The compounds of the present invention may themselves be active and/or act as prodrugs which convert in vivo to active dipyridinium compounds of the invention. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001).

Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

More specifically, the compounds of the present invention wherein α is polyethylene glycolyl may themselves be active and/or act as carrier prodrugs. Carrier prodrugs are drug compounds that contain a transport moiety, e.g., that improve uptake and/or localized delivery to a site(s) of action. It is believed that that the pegylated dipyridium compounds of the present invention undergo cleavage of the covalently bonded polyethylene glycol group to release an active dipyridinium moiety within the confines of the tumour environment.

Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C_{,} ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed,

Pharmaceutically acceptable solvates in accordance with the invention include those wherein the solvent of crystallization may be isotopically substituted, e.g. D₂O, d₆-acetone, d₆-DMSO.

### Synthesis

The compounds of the invention may be synthesized by the general synthetic routes below, specific examples of which are described in more detail in the Examples.

Compounds of formula I wherein α is polyethylene glycolyl may be prepared according to Scheme 1.

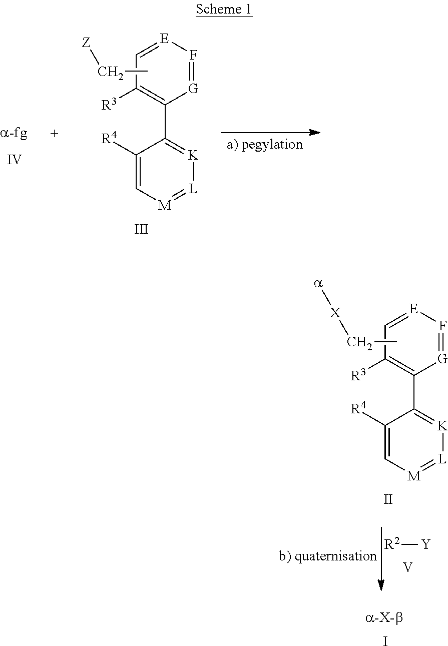

wherein X, Y, R³ and R⁴ are as defined in respect of a compound of formula I; E, F, G, K, L and M are each independently selected from CR¹ and N with the proviso that E, F or G is N and K, L or M is N and only one of E, F and G is N and only one of K, L and M is N; R¹ and R² are as defined in respect of a compound of formula (I); Z is halo (e.g. Br), OH, NH₂; fg is a functionalised group, in particular NH₂, COOH, OH, mesylate, CHO.

Compounds of formula III may be prepared according to Scheme 1a.

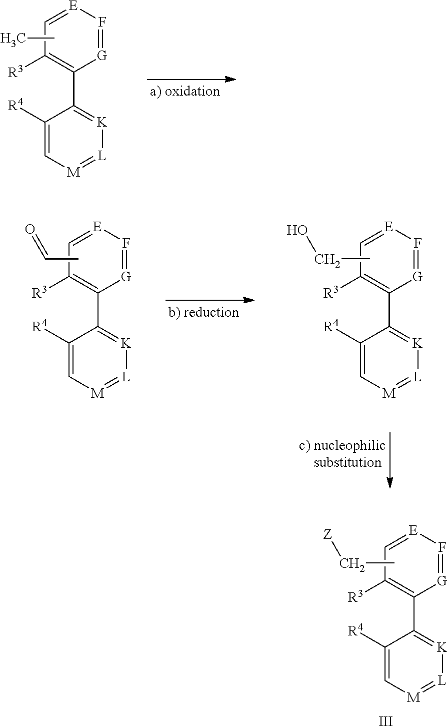

Alternatively, Compounds of formula III may be prepared according to Scheme 1b.

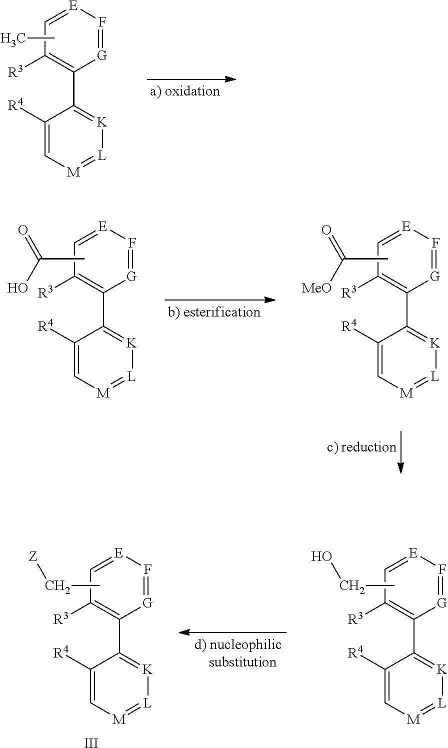

Compounds of present invention wherein α is a bifunctional polyethylene glycolyl may be prepared according to Scheme 1c.

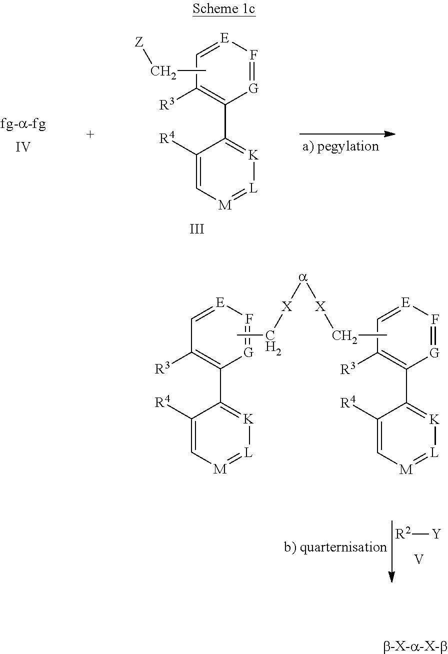

wherein X, Y, R³ and R⁴; E, F, G, K, L and M; R¹ and R²; Z and fg are as defined with respect to Scheme 1.

Compounds of formula I wherein α is H may be prepared according to Scheme 2.

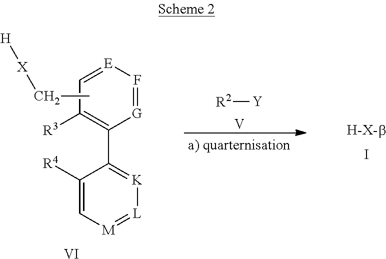

wherein X, R³ and R⁴ are as defined in respect of a compound of formula (I); E, F, G, K, L and M are each independently selected from CR¹ and N with the proviso that E, F or G is N and K, L or M is N and only one of E, F and G is N and only one of K, L and M is N; R¹ and R² are as defined in respect of a compound of formula (I).

Compounds of formula (VI) may be prepared by analogous processes to the compounds of formula (III)

The above general schemes may be used to prepare compounds of the present invention. The desired specific compounds can be prepared by selecting the appropriate starting materials, reactants and reaction conditions.

The starting materials and reagents in the above scheme are all either available commercially or can be prepared following literature precedents.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage). Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers. Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

As a further aspect of the present invention, there is also provided a process for the preparation of compounds of formula I or a pharmaceutically acceptable salt or solvate thereof.

According to a further aspect of the invention there is provided a process of preparing a compound of the present invention or a pharmaceutically acceptable salt or solvate thereof comprising the step of:

a) wherein a is polyethylene glycolyl, quaternisation of a compound of formula II

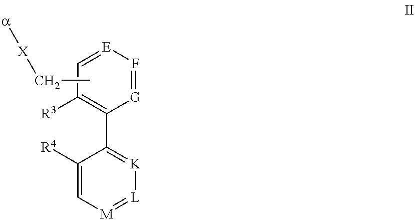

by reacting with a compound of formula V (R²—Y) under conventional quaternisation conditions wherein X, Y, R³ and R⁴ are as defined in respect of a compound of formula (I); E, F, G, K, L and M are each independently selected from CR¹ and N with the proviso that E, F or G is N and K, L or M is N and only one of E, F and G is N and only one of K, L and M is N; R¹ and R² are as defined in respect of a compound of formula I; or

b) wherein a is H, quaternisation of a compound of formula VI

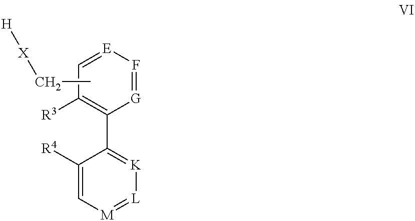

by reacting with a compound of formula V (R²—Y) under conventional quaternisation conditions,
wherein X, Y, R³ and R⁴ are as defined in respect of a compound of formula (I); E, F, G, K, L and M are each independently selected from CR¹ and N with the proviso that E, F or G is N and K, L or M is N and only one of E, F and G is N and only one of K, L and M is N; R¹ and R² are as defined in respect of a compound of formula (I).

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known to those skilled in the art.

The agents of the invention act to generate systemic reactive oxygen species (ROS) in particular at sites of pathologically proliferating tissue, or other sites where it may be therapeutically advantageous. Reactive oxygen species are activated forms of molecular oxygen, which can destroy all classes of biological molecules, including DNA, protein, lipids, sterols and vitamins¹ (Lee-Ruff, The Organic Chemistry of Superoxide, Chem Soc Rev, 1977, 6:195-214; Bryant Miles, Oxygen Metabolism and Oxygen Toxicity. Feb. 26, 2003. www.tamu.edu/classes/bich/bmiles/lectures/; Keyer K, Imlay J A. Superoxide accelerates DNA damage by elevating free-iron levels, Proc Natl Acad Sci USA. 1996 Nov. 26; 93(24):13635-40; Winterbourn C C, Kettle A J, Radical-radical reactions of superoxide: a potential route to toxicity, Biochem Biophys Res Commun. 2003 Jun. 6; 306(3):729-36. Review; Babior B M, Superoxide: a two-edged sword, Braz J Med Biol Res. 1997 February; 30(2):141-55. Review; Kang Dongchon, Mitochondria as a Target of Medicinal Chemistry, Current Medicinal Chemistry, Volume 10, Number 23, December 2003, pp. i-ii (1)).

They are constantly produced during normal aerobic metabolism, but are kept in harmless check by defence systems such as cellular compartmentalisation and by enzymes such as superoxide dismutase and catalyase that inactivate these species. With increased production, however, such defences are inadequate and the reactive oxygen species cause cell death and tissue destruction (necrosis). Controlled use of reactive oxygen species can be therapeutically useful however. Examples in which they are used include PUVA (Psoralen Ultra Violet A light) for psoriasis and other skin diseases (Diffey B., The contribution of medical physics to the development of psoralen photochemotherapy (PUVA) in the UK: a personal reminiscence, Phys Med. Biol. 2006 Jul. 7; 51(13):R229-44) and Photodynamic therapy (Rodriguez E, Baas P, Friedberg J S., Innovative therapies: photodynamic therapy, Thorac Surg Clin. 2004 November; 14(4): 557-66) which is used to treat cancer and ‘benign’ conditions like age related macular degeneration Both PUVA and Photodynamic therapy generate singlet oxygen in situ.

In other work, Fang et al. (Fang J, Sawa T, Akaike T, Maeda H., Tumor-targeted delivery of polyethylene glycol-conjugated D-amino acid oxidase for antitumor therapy via enzymatic generation of hydrogen peroxide, Cancer Res. 2002 Jun. 1; 62(11): 3138-43) have shown that hydrogen peroxide generated in situ significantly suppressed the growth of tumour xenografts in mice. Yoshikawa et al. (Yoshikawa T, Kokura S, Tainaka K, Naito Y, Kondo M., A novel cancer therapy based on oxygen radicals, Cancer Res. 1995 Apr. 15; 55(8):1617-20) showed that superoxide generated in situ significantly suppressed the growth of tumour xenografts in rabbits and Stamler and colleagues report clinical case histories in U.S. Pat. No. 6,231,894 where superoxide brought about regression of a melanoma and oral squamous cell carcinoma.

The above treatments are important examples of the therapeutic power of reactive oxygen species but are limited to local use. The compounds of the invention are designed for systemic use and are expected to generate reactive oxygen species in a controlled manner, that target pathological tissue without affecting normal tissue. An agent of this kind is not constrained by the locality of the pathological proliferating tissue.

Whilst not bound by theory, the compounds of the invention have a similar capacity for accepting electrons (similar reduction potential) as do the flavanoid proteins of cell metabolism and can decouple the electron flow in normal biological reduction pathways by abstracting one electron and transferring it to molecular oxygen [O₂], forming superoxide [O₂], in the process (Thorneley R N., A convenient electrochemical preparation of reduced methyl viologen and a kinetic study of the reaction with oxygen using an anaerobic stopped-flow apparatus, Biochim Biophys Acta. 1974 Mar. 26; 333(3):487-96).

An example of recycling by the dipyridinium redox agent diquat is shown in Scheme A, where NADPH is the electron donor

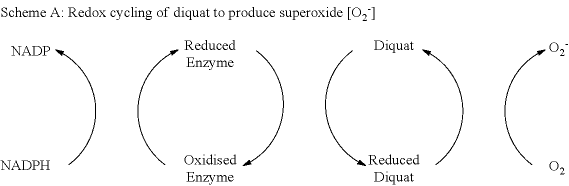

This class of redox agents generate superoxide catalytically by continuously cycling between oxidised and reduced states. Dipyridinium salts are very effective electron shunting compounds in vivo because of their similar reduction potential to the flavanoid proteins of cell metabolism and two, diquat and paraquat, are widely-used herbicides, through their capacity to produce lethal levels of superoxide in plants (Hassan H M, Fridovich I., Intracellular production of superoxide radical and of hydrogen peroxide by redox active compounds, Arch Biochem Biophys. 1979 September; 196(2):385-95; Mira, D., Brunk, U., Boveris, A., and Cadenas, E., One-electron transfer reactions of diquat radical to different reduction intermediates of oxygen, Free Rad. Biol. Med. 5, 155-163 1988; Bus, J. S., Aust, S. D., and Gibson, J. E., Superoxide-and singlet oxygen-catalyzed lipid peroxidation as a possible mechanism for paraquat (methyl viologen) toxicity, Biochem. Biophys. Res. Commun. 58, 749-755 1974).

Several different enzymes like NADPH oxidoreductase, xanthine oxidase, and inducible nitric oxide synthase can reduce dypyridinium salts (Margolis, A. S., Porasuphatana, S., and Rosen, G. M., Role of paraquat in the uncoupling of nitric oxide synthase, Biochim. Biophys. Acta 1524, 253-257; Kitazawa, Y., Matsubara, M., Takeyama, N., and Tanaka, T., The role of xanthine oxidase in paraquat intoxication, Arch. Biochem. Biophys. 288, 220-224; Kalinowski L, Malinski T, Endothelial NADH/NADPH-dependent enzymatic sources of superoxide production: relationship to endothelial dysfunction, Acta Biochim Pol. 2004; 51(2):459-69. Review.; Clejan L A, Cederbaum A I, Stimulation by paraquat of microsomal and cytochrome P-450-dependent oxidation of glycerol to formaldehyde, Biochem J, 1993 Nov. 1; 295 (Pt 3):781-6; Stuehr D, Pou S, Rosen G M, Oxygen reduction by nitric-oxide synthases, J Biol. Chem. 2001 May 4; 276(18):14533-6; Day B J, Patel M, Calavetta L, Chang L Y, Stamler J S, A mechanism of paraquat toxicity involving nitric oxide synthase, Proc Natl Acad Sci USA. 1999 Oct. 26; 96(22):12760-5).

These enzymes are present in the cytoplasm, the mitochondria, microsomes and blood vessel endothelia, so superoxide may be generated at many different points in a cell and its supporting blood vessels (Van Heerebeek L, Meischl C, Stooker W, Meijer C J, Niessen H W, Roos D., NADPH oxidase(s): new source(s) of reactive oxygen species in the vascular system? J Clin Pathol. 2002 August; 55(8):561-8. Review; Yumino K, Kawakami I, Tamura M, Hayashi T, Nakamura M., Paraquat-and diquat-induced oxygen radical generation and lipid peroxidation in rat brain microsomes, J Biochem (Tokyo). 2002 April; 131(4):565-70.

These multiple points of action will be beneficial in the treatment of cancer.

Whilst not bound by theory, the compounds of the invention produce reactive oxygen species catalytically, in a process that does not sequester these compounds immediately. A skilled person will appreciate that catalytic generation greatly facilitates the therapeutic potential of this ROS therapy, in comparison to the use of compounds which generate ROS in approximately equimolar quantities.

Having regard to their generation of systemic ROS and inhibition of cellular proliferation, the compounds of the present invention, hereinafter alternately referred to as “agents of the invention”, are useful in the treatment of a disease or disorder characterised by pathologically proliferating cells, particularly cancer

A disease or disorder characterised by pathologically proliferating cells is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, melanoma and non-melanomatous skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), gastrointestinal stromal tumor, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cartilidge, or bone, cancer of the urethra, cancer of the penis, prostate cancer, testicular cancer testicular lymphoma, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In one embodiment of the present invention the cancer is lung cancer (NSCLC and SCLC), melanoma, cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, cancer of the thyroid gland, cancer of the parathyroid gland, pancreatic cancer, prostate cancer, neoplasms of the central nervous system (CNS), primary CNS lymphoma, non hodgkins's lymphoma, or spinal axis tumors, or a combination of one or more of the foregoing cancers.

In a particular embodiment, the cancer is lung cancer (NSCLC and SCLC), melanoma, cancer of the head or neck, ovarian cancer, breast cancer, prostate cancer, colon cancer, or renal cell carcinoma.

In another embodiment, said disease or disorder characterised by pathologically proliferating cells is a benign proliferative disease, including, but not limited to, psoriasis, benign prostatic hypertrophy or restinosis

Treatment in accordance with the invention may be symptomatic or prophylactic.

Thus in a further aspect the invention includes an agent of the invention for use as a pharmaceutical.

Therefore according to a further aspect, the invention provides an agent of the invention for treating or preventing a disease or disorder characterised by pathologically proliferating cells.

Therefore according to a further aspect, the invention provides the use of an agent of the invention in the manufacture of a medicament for the treatment or prevention of a disease or disorder characterised by pathologically proliferating cells.

Therefore according to a further aspect, the invention provides a method for preventing or treating a disease or disorder characterised by pathologically proliferating cells in which an effective amount of an agent of the invention is administered to a patient in need of such treatment.

In accordance with the foregoing, the invention provides a method for preventing or treating a disease or disorder characterised by pathologically proliferating cells, particularly cancer, which comprises administering to a subject, particularly a human subject, in need thereof a compound of the present invention.

In another embodiment, the invention provides a compound of the present invention for preventing or treating a disease or disorder characterised by pathologically proliferating cells, particularly cancer.

In another embodiment, the invention provides the use of a compound of the present invnetion in the manufacture of a medicament for the prevention or treatment of a disease or disorder characterised by pathologically proliferating cells, particularly cancer.

Cell culture studies have shown that the dipyridinium salt diquat is toxic in vitro with an LC 50 in the 40-180 micromolar range depending on the cell studied (Suleiman S A, Stevens J B, Bipyridylium herbicide toxicity: effects of paraquat and diquat on isolated rat hepatocytes. J Environ Pathol Toxicol Oncol. 1987 January-February; 7(3):73-84; Osburn W O, Wakabayashi N, Misra V, Nilles T, Biswal S, Trush M A, Kensler T W. Nrf2 regulates an adaptive response protecting against oxidative damage following diquat-mediated formation of superoxide anion. Arch Biochem Biophys. 2006 Oct. 1; 454(1):7-15.

This is comparable to the LC 50 value for the widely used anticancer agents Carboplatin and Paclitaxel (Georgiadis M S, Russell E K, Gazdar A F, Johnson B E. Paclitaxel cytotoxicity against human lung cancer cell lines increases with prolonged exposure durations Clin Cancer Res. 1997 March; 3(3): 449-54). Modified mouse fibroblasts Nrf2−/−, which cannot mount a protective response to reactive oxygen species have approximately a fourfold increase in sensitivity to diquat, compared to normal unmodified [wildtype] cells.

Free diquat salt is toxic in large doses, though less so than many anticancer agents currently used, at least by LD50 assays (Tablet). In mammals it mainly affects liver, kidney and gut, and in humans, neurological tissue. Dipyridinium salts therefore have the potential to be used therapeutically in the treatment of cancer.

TABLE 1
LD50 values of diquat and selected anticanceragents
Oral Toxicities of selected anti cancer agents

| Compound | LD50 dog mg/kg | LD50 rat mg/kg |
|---|---|---|
| Cisplatin | 2.5iv | 25 |
| Doxorubicin | 2.0 | 12.5 |
| Methotrexate | 120 | 180-320 |
| Diquat | 100-200 | 231 |

Whilst not bound by theory, the compounds of the present invention reduce or eliminate systemic exposure (and potential toxicity of the free dipyridinium compounds) via the preferential uptake of the drug to the tumour through linkage to polyethylene glycol or other appropriate polymer and the Enhanced Permeability and Retention (EPR) property of tumours (Iyer A K, Khaled G, Fang J, Maeda H. Exploiting the enhanced permeability and retention effect for tumor targeting, Drug Discov Today. 2006 September; 11(17-18):812-8). This is a physiological phenomenon, which favours the accumulation of high molecular weight compounds over low molecular weight compounds, through ‘passive’ targeting. The bodily distribution of low molecular weight compounds can therefore be completely changed by simple procedures that turn them into high molecular weight compounds, for example by binding them to a polymer. The principle of enhanced permeability and retention is further demonstrated in the preclinical studies of Fang et al. (ibid).

The enhanced permeability and retention of compounds in tumours results from “passive” accumulation in the tumour bulk, as distinct from “active” targeting of specific sites in cells and receptors within the tumour. It is believed that EPR arises because blood vessels within the tumour vessels are tortuous and leaky, which combined with similarly disorganised draining lymphatics, results in a combined effect which causes reduced blood flow or stasis. A drug compound is transported through a tumour via diffusion and therefore in this environment high molecular weight compounds are preferentially retained. Thus, covalent addition of an inert polymer may be used to enhance the tumour specificity of a drug compound of interest via the EPR effect.

Use of polyethylene glycol as the inert polymer confers many additional potential benefits. In particular, pegylated compounds are not immunogenic, so sequestration by the reticulo-endothelial system, in particular the liver and spleen, is prevented and may result in an enhanced safety profile and longer circulation time.

A longer circulation time may improve bioavailability and cytotoxicity of the pegylated dipyridinium compounds of the present invention compared to short circulation times, rapid metabolism, and low tumor uptakes that are typically characteristic of small molecular weight compounds

The toxicity of dipyridinium salts, both novel compounds described herein and those described in the prior art, may therefore be sufficiently minimised by pegylation to render these compounds pharmaceutically acceptable even in situations where inherent toxicity would likely have otherwise prevented this. The xenograft studies exemplified in Example 6 (see FIG. 6) show that compounds of the invention can be administered to tumour bearing mice without any loss of weight, whereas cisplatin, a standard anticancer agent, causes significant weight loss.

It is therefore believed that compounds of present invention may be administered in a standard clinical setting, for example in district general hospitals, without the procedural restrictions required by the use of conventional cytotoxics.

In another aspect, the present invention provides a pharmaceutical composition comprising a compound of the present invention and a pharmaceutically acceptable carrier.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of the present invention and at least one other therapeutic agent as a combined preparation for simultaneous, separate or sequential use in therapy. In one embodiment, the therapy is the treatment of a disease or disorder characterised by pathologically proliferating cells. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable excipient, as described above.

A skilled person will appreciate that a compound of the invention may be administered to a subject, particularly a human subject, wherein the subject is being treated with surgery or radiotherapy for a disease or disorder characterised by pathologically proliferating cells. A compound of the invention may also be administered to a subject, particularly a human subject, wherein the subject has previously (e.g. within 24 hours) been treated with surgery or radiotherapy for a disease or disorder characterised by pathologically proliferating cells, A subject, particularly a human subject, may also be treated with surgery or radiotherapy for a disease or disorder characterised by pathologically proliferating cells wherein a compound of the invention has previously (e.g. within 24 hours) been administered to a subject,

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of an agent of the invention for treating a disease or disorder characterised by pathologically proliferating cells, wherein the medicament is prepared for administration with another therapeutic agent. The invention also provides the use of another therapeutic agent for treating a disease or disorder characterised by pathologically proliferating cells, wherein the medicament is administered with a compound of the invention.

The invention also provides a compound of invention for use in a method of treating a disease or disorder characterised by pathologically proliferating cells, wherein the compound of the invention is prepared for administration with another therapeutic agent. The invention also provides another therapeutic agent for use in a method of treating a disease or disorder characterised by pathologically proliferating cells, wherein the other therapeutic agent is prepared for administration with a compound of formula (I). The invention also provides a compound of invention for use in a method of treating a disease or disorder characterised by pathologically proliferating cells, wherein the compound of the invention is administered with another therapeutic agent. The invention also provides another therapeutic agent for use in a method of treating a disease or disorder characterised by pathologically proliferating cells, wherein the other therapeutic agent is administered with a compound of the invention.

The invention also provides the use of a compound of the invention for treating a disease or disorder characterised by pathologically proliferating cells, wherein the subject has previously (e.g. within 24 hours) been treated with another therapeutic agent. The invention also provides the use of another therapeutic agent for treating a disease or disorder characterised by pathologically proliferating cells, wherein the subject has previously (e.g. within 24 hours) been treated with a compound of the invention.

In one embodiment, the other therapeutic agent is an anti-tumour agent selected from the group consisting of antiproliferative agents, kinase inhibitors, angiogenesis inhibitors, growth factor inhibitors, cox-I inhibitors, cox-II inhibitors, mitotic inhibitors, alkylating agents, antimetabolites, intercalating antibiotics, growth factor inhibitors, radiation, cell cycle inhibitors, enzymes, topoisomerase inhibitors, biological response modifiers, antibodies, cytotoxics, anti-hormones, statins, anti-androgens and photochemotherapy agents.

Accordingly, the invention includes as a further aspect a combination of an agent of the invention with an anti-tumour agent selected from the group consisting of antiproliferative agents, kinase inhibitors, angiogenesis inhibitors, growth factor inhibitors, cox-I inhibitors, cox-II inhibitors, mitotic inhibitors, alkylating agents, antimetabolites, intercalating antibiotics, growth factor inhibitors, radiation, cell cycle inhibitors, enzymes, topoisomerase inhibitors, biological response modifiers, antibodies, cytotoxics, anti-hormones, statins, anti-androgens and photochemotherapy agents.

In one embodiment of the present invention the anti-tumor agent used in conjunction with a composition of the present invention is an anti-angiogenesis agent, kinase inhibitor, pan kinase inhibitor or growth factor inhibitor.

Preferred pan kinase inhibitors include SU-11248 (sutinib malate), described in U.S. Pat. No. 6,573,293 (Pfizer Inc).

Anti-angiogenesis agents, include but are not limited to the following agents, such as EGF inhibitors, EGFR inhibitors, VEGF inhibitors, VEGFR inhibitors, TIE2 inhibitors, IGF1 R inhibitors, COX-II (cyclooxygenase II) inhibitors, MMP-2 (matrix-metalloprotienase 2) inhibitors, and MMP-9 (matrix-metalloprotienase 9) inhibitors. Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc.), AG13736 (axitinib, Pfizer Inc.), ZD-6474 (AstraZeneca), AEE788 (Novartis), AZD-2171), VEGF Trap (Regeneron/Aventis), Vatalanib (also known as PTK-787, ZK-222584: Novartis & Schering A G), Macugen (pegaptanib octasodium, NX-1838, EYE-001, Pfizer Inc./Gilead/Eyetech), IM862 (Cytran Inc. of Kirkland, Wash., USA); and Angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposes. Particularly preferred VEGF inhibitors include CP-547,632, AG13736, Vatalanib, Macugen and combinations thereof.

Other antiproliferative agents that may be used with the compositions of the present invention include inhibitors of the enzyme farnesyl protein transferase and inhibitors of the receptor tyrosine kinase PDGFr.

PDGRr inhibitors include but are not limited to those disclosed in international patent application publication number WO01/40217, published Jul. 7, 2001 and international patent application publication number WO2004/020431, published Mar. 11, 2004, the contents of which are incorporated in their entirety for all purposes. Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts). GARF inhibitors useful in the practice of the present invention are disclosed in U.S. Pat. No. 5,608,082 which is incorporated in its entirety for all purposes.

Examples of useful COX-II inhibitors which can be used in conjunction with compounds of the invention described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2(4-Ethmryphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes

Other useful inhibitors as anti-tumor agents used in conjunction with compositions of the present invention include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents used in conjunction with a composition of the present invention include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Preferred EGFr inhibitors include Iressa, Erbitux, Tarceva and combinations thereof. Other anti-tumor agents include those selected from pan erb receptor inhibitors or ErbB2 receptor inhibitors, such as CP-724,714 (Pfizer, Inc.), CM 033 (canertinib, Pfizer, Inc.), Herceptin (trastuzumab, Genentech Inc.), Omitarg (2C4, pertuzumab, Genentech Inc.), TAK-165 (Takeda), GW-572016 (lonafamib, GlaxoSmithKline), GW-282974 (GlaxoSmithKline), EKB-569 (Wyeth), PKM 66 (Novartis), dHER2 (HER2 Vaccine, Corixa and GlaxoSmithKline), APC8024 (HER2Vaccine, Dendreon), anti-HER2/neu bispecific antibody (Decof Cancer Center), B7.her2.1gG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof. Preferred erb selective anti-tumor agents include Herceptin, TAK-165, CP-724,714, ABX-EGF, HER3 and combinations thereof. Preferred pan erbb receptor inhibitors include GW572016, CM 033, EKB-569, and Omitarg and combinations thereof.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antlsoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE)1 ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may also be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpimase), BEC2 (mitumoab), Xcytrin (motexafin gadolinium) and combinations thereof. Further anti-tumor agents may selected from the following agents, CeaVac (CEA), NeuTrexin (trimetresate glucuronate) and combinations thereof. Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof. Additional anti-tumor agents may selected from the following agents, RSR13 (efaproxiral), Cotara (1311 chTNT 1/b), NBI-3001 (IL-4) and combinations thereof. Additional anti-tumor agents may selected from the following agents, Canvaxin, GMK vaccine, PEG Interon A, Taxoprexin (DHA/paciltaxel) and combinations thereof.

Other anti-tumor agents include Pfizer's MEK1/2 inhibitor PD325901, Array Biopharm's MEK inhibitor ARRY-142886, Bristol Myers' CDK2 inhibitor BMS-387,032, Pfizers CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc/Aton Pharmaceuticals) and combinations thereof. Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in conjunction with a composition of the present invention as described herein.

The invention also contemplates the use of the compositions of the present invention together with hormonal therapy, including but not limited to, exemestane (Aromasin, Pfizer Inc.), leuprorelin (Lupron or Leuplin, TAP/Abbottriakeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex (R)(4′-cyano-3-(4-fluorophenylsulphonyl)-2-hydroxy-2-methyl-3′-(trifluoromethyl) propionanilide, bicalutamide) and combinations thereof.

Further, the invention provides a composition of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, lressa, Gleevec, Taxotere and combinations thereof.

The following topoisomerase I inhibitors may be utilized as anti-tumor agents: camptothecin; irinotecan HCl (Camptosar); edotecarin; orathecin (Supergen); exatecan (Daiichi); BN-80915 (Roche); and combinations thereof. Particularly preferred toposimerase II inhibitors include epirubicin (Ellence).

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof. Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, LIFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexoi, raltitrexed, triapine, trimetrexate, vidarabine, vincristine, vinorelbine; or for example, one of the preferred anti-metaboiites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, vairubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Eilence), etoposide, exatecan, gimatecan, lurtotecan, mitoxantrone, pirarubicin, pixantrone, rubitecan, sobuzoxane, SN-38, tafluposide, topotecan, and combinations thereof. Preferred cytotoxic topoisomerase inhibiting agents include one or more agents selected from the group consisting of camptothecin, 10-hydroxycamptothecin, 9-aminocamptothedn, irinotecan HCl (Camptosar), edotecarin, epirubicin (Eilence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, ientinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoV AX-CL, sargramostim, tasonermin, tecleukin, thymalasin, tositumomab, Virulizin, 2-100, epratuzumab, mitumomab, oregovomab, pemtumomab (Y-muHMFGI), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, ubenimex and combinations thereof.

Other anticancer agents include alitretinoin, ampligen, atrasentan bexarotene, bortezomib. Bosentan, calcitriol, exisuiind, finasteride. fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof. Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof. Other antitumor agents include mitoxantrone, 1-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additionally, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those. described in International Patent Application No. WO 2002/053596, which is herein incorporated by reference in its entirety. Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention, statins may be used in conjunction with a composition of the present invention. Statins (HMG-CoA reducatase inhibitors) may be selected from the group consisting of Atorvastatin (Lipitor, Pfizer Inc.), Pravastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof. In a preferred embodiment the statin is selected from the group consisting of Atovorstatin and Lovastatin, derivatives and combinations thereof. Other agents useful as anti-tumor agents include Caduet.

In one embodiment of the invention, the compositions of the present invention may be used in conjunction with photochemotherapy agents which are used to generate reactive oxygen species locally. Examples of photochemotherapy agents include palladium bacteriophephorbide (TOOKAD) used in photodynamic therapy; psoralen, 8-methoxypsoralen/methoxsalen (Oxsoralen-Ultra®, 8-MOP®, Oxsoralen®, Uvadex®), 4,5,8-trimethylpsoralen/trioxsalen (Trisoralen®), used in PUVA (Psoralen Ultra Violet A light); UVAR or UVAR® XTS™ Photopheresis System (Therakos, Inc., Exton, Pa.): Theraflex ECP® (Macopharma); CobeSpectra+Photo Immune System UVA PIT (Med Tech Solution); photosensitizers such as calcipotriene, tazarotene, chrysarobin and its synthetic derivative anthralin/1,8-dihydroxy-9-anthrone/dithranol (Drithocreme®); firefly (Photinus pyralis) luciferase used in (BioLuminescence Activated Destruction (BLADe)); erythrosin B (EB); erythrosine sodium; m-tetra(hydroxyphenyl)chlorin (m-THPC)/temoporfin (Foscan®, Biolitec AG); porphyrins such as d-aminolevulinic acid (d-ALA) (Levulan Kerastick®; DUSA Pharmaceuticals, Inc.), 5-ALA methylesther (MLA/M-ALA) (Metvix®; PhotoCure ASA), 5-ALA benzylesther (Benzvix®); 5-ALA hexylesther (Hexvix®), tin ethyl etiopurpurin (SnET2)/Sn etiopurpurin/rostaporfin (Photrex®, Purlytin®; Miravant MedicalTechnologies, boronated protoporphyrin (BOPP®), 2-(1-hexyloxyethyl)-2-divinyl pyropheophorbide-a (HPPH) (Photochlor®; Rosewell Park Cancer Institute), texaphyrins including europium texaphyrin (Eu-Tex), dysprosium texaphyrin (Dy-Tex), manganese texaphyrin (Mn-Tex), lutetium texaphyrin/PCI-0123 (Lu-Tex®, Lutex®, Lutrin®), motexafin lutetium (MLu)/lutetium(III) texaphyrin (Lu-Tex) (Antrin®, Lutrin®, Optrin®; Pharmacyclics Inc.), motexafin gadolinium (MGd)/PCI-0120 (Xcytrin®; source: Pharmacyclics Inc.) phthalocyanine-4 (Pc 4), taporfin sodium/NPe6/mono-L-aspartyl chlorin e6/taporfin sodium/LS11 (Talaporfin®; Light ScienceCorporation), benzoporphyrin derivative-monoacid ring A (BPD-MA)/verteporfin (Visudyne®, Novartis Pharmaceuticals), hematoporphyrin derivative (HpD) partially purified, porfimer sodium (Photofrin®; Axcan Pharma, Inc.), dihematoporphyrin ethers (DHE), photosan-3 (PS-3), photofrin-II, meso-tetrakis-phenylporphyrin (TPP) and tetraphenylporphinesulfonate (TPPS4)

The compounds of the present invention have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

As outlined hereinabove, the dipyridinium salts of the present invention have many advantages in comparison to conventional anticancer agents. Some are briefly stated below:

The dipyridinium salts of the invention may work synergistically with cell metabolic processes to generate superoxide All metabolically-active cancers, and which are growing, may be treatable, because the compound acts at the ‘fundamental’ level of cell metabolism. This may allow the treatment of advanced and metastatic cancers, including those which have escaped control and which are resistant and refractory to other therapeutics.

The specificity of the compounds for cancer may be increased by the Enhanced permeability and retention [EPR] effect, with systemic exposure an potentially toxicity limited in the process.

The specificity of pegylated dipyridinum salts for the tumour may be further increased by the catalytic production of reactive oxygen species which is differentially magnified in regions of high drug-concentration,

Tumour specificity is further enhanced by the reduction of dipyridinium salt by inducible nitric oxide synthase, which is over expressed in some tumours including colorectal tumours

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329).

Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to at least partially alleviating, inhibiting, preventing and/or ameliorating a disease or disorder characterised by pathologically proliferating cells. In another non-limiting embodiment, the term “a therapeutically effective amount” refers to the amount of the compound of the present invention that, when administered to a cell, or a tissue, or a non-cellular biological material, or a medium, is effective to at least partially reducing cellular proliferation.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The invention is illustrated by the following Examples.

### EXAMPLES

Referring to the examples that follow, compounds of the preferred embodiments are synthesized using the methods described herein, or other methods, which are known in the art.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

### General Conditions:

The following examples are intended to illustrate the invention and are not to be construed as being limitations thereon. Temperatures are given in degrees centigrade. If not mentioned otherwise, all evaporations are performed under reduced pressure. The structure of final products, intermediates and starting materials is confirmed by standard analytical methods, e.g., microanalysis and spectroscopic characteristics, e.g., MS, IR, NMR. Abbreviations used are those conventional in the art. If not defined, the terms have their generally accepted meanings.

### ABBREVIATIONS

bs broad singlet
d doublet
d(n) day (number of day)
DCM dichloromethane
DMF N,N-dimethylformamide
DMSO dimethylsulfoxide
DTE ethane ditriflate (2-(trifluoromethylsulfonyloxy)ethyl trifluoromethanesulfonate)
h hour(s)
i.p. intraperitoneal
i.v intraveneous
MeOH methanol
mPEG5000-methoxypolyethyleneglycolyl group of average molecular weight 5000 daltons
MS-ES+ mass spectrometry-electrospray
m multiplet
min minutes
ml milliliter(s)
m/z mass to charge ratio
NMR nuclear magnetic resonance
PEG3400 polyethyleneglycolyl group of average molecular weight 3400 daltons
ppm parts per million
s singlet
SWV square wave voltammagram
t triplet
TEA triethylamine
TFA trifluoroacetic acid
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

Example compounds of the present invention include:

### Example 1

### Compound B

### 3-(hydroxymethyl)-6,7-dihydrodipyrido[1,2-a:2′,1′-c]pyrazine-5,8-diium bistrifluoromethanesulfonate

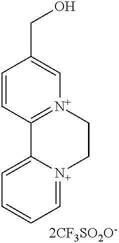 [SMILES: OCc1ccc2[n+](c1)CC[n+]1ccccc1-2]

To a solution of 5-hydroxymethyl-2,2-bipyridyl (Preparation 1, 90 mg, 0.48 mmol) in anhydrous CH₂Cl₂ (5 mL) was added DTE (Preparation 2, 237 mg, 0/3 mmol) and the solution was stirred under nitrogen at room temperature overnight.

The resultant precipitate was collected by filtration and washed with anhydrous CH₂Cl₂ to afford the title compound as an off-white solid (136 mg, 55% yield).

¹H NMR (DMSO-d₆, 500 MHz) δ 4.88 (d, 2H), 5.26 (m, 4H), 6.12 (t, 1H), 8.44 (m, 1H), 8.82 (dd, 1H), 8.95 (m, 1H), 9.08 (dd, 2H), 9.30 (m, 2H).

### Example 2

### Compound C

### 3-((2-(2-methoxy-ethoxy)(ethoxy)ₙ)methyl)-6,7-dihydrodipyrido[1,2-a:2′,1′-c]pyrazine-5,8-diium bistrifluoromethanesulfonate

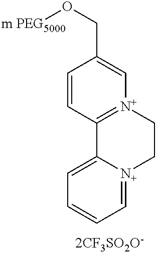

To a solution of 5-methyl-2,2-bipyridyl mPEG (5000) ether (Preparation 5, 2.70 g, 0.52 mmol) in anhydrous CHCl₃ (270 mL) was added DTE (Preparation 2, 255 mg, 0.78 mmol) and the solution was heated at reflux under nitrogen overnight.

The reaction mixture was filtered and the filtrate concentrated in vacuo to ca. 4 mL volume and the residue was slurried in anhydrous ether (100 mL). The precipitate was collected by filtration to afford the title compound as a pale pink solid (2.25 g, 78% yield).

¹H NMR (CDCl₃, 500 MHz) δ 3.37 (s, 3H), 3.64 (m, ca. 452H), 4.82 (s, 2H), 4.97 (m, 2H), 5.54 (m, 2H), 7.75 (m, 1H), 8.31 (m, 1H), 8.40 (m, 1H), 8.56 (m, 2H), 8.99 (m, 2H).

UV-VIS λₘₐₓ 288 nm.

### Example 3

### Compound D

### 3-(((2-(2-methoxyethoxy)(ethoxy)ₙ)ethyl) hexane-1,6-diyldicarbamate)methyl)-6,7-dihydrodipyrido[1,2-a:2′,1′-c]pyrazine-5,8-diium bistrifluoromethanesulfonate

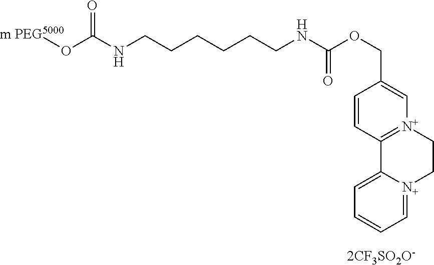

To a solution of mPEG (5000) extended chain carbamate (Preparation 6, 1.00 g, 0.19 mmol) in anhydrous CHCl₃ (150 mL) was added DTE (Preparation 2, 91 mg, 0.28 mmol) and the solution was heated at reflux under nitrogen overnight.

The reaction mixture was filtered and the filtrate concentrated in vacuo to ca. 2 mL volume and the residue was slurried in anhydrous ether (50 mL). The precipitate was collected by filtration to afford the title compound as an off-white solid (790 mg, 75% yield).

¹H NMR (CDCl₃, 500 MHz) δ 1.24-1.52 (m, 12H), 3.38 (s, 3H), 3.64 (m, ca 452H), 4.12 (bs, 1H), 4.68 (bs, 1H), 4.88 (m, 2H), 5.27 (s, 2H), 5.52 (m, 2H), 7.32 (m, 1H), 7.89 (m, 1H), 8.23 (m, 1H), 8.48 (m, 1H), 8.60 (m, 1H), 8.99 (m, 1H), 9.04 (m, 1H).

UV-VIS λₘₐₓ 308 nm.

### Example 4

### Compound E

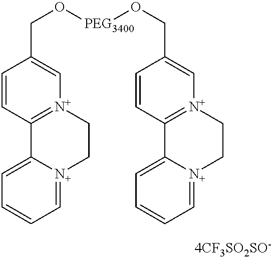

To a solution of PEG (3400) diether (Preparation 8, 100 mg, 0.03 mmol) in anhydrous CHCl₃ (15 mL) was added DTE (38 mg, 0.12 mmol) and the solution was heated at reflux under nitrogen overnight.

The reaction mixture was concentrated in vacuo to 0.5 mL and diethyl ether (30 mL) was added. The resultant precipitate was collected by filtration, washed with diethyl ether (8 mL) and dried to afford a very pale pink solid (1.79 g, 85% yield). filtered and the filtrate concentrated in vacuo to ca. 4 mL volume and the residue was slurried in anhydrous ether (100 mL). The precipitate was collected by filtration to afford the title compound as a pale pink solid (85 mg, 73% yield).

¹H NMR (DMSO-d, 500 MHz) δ 3.40-3.80 (m, ca. 308H), 4.88 (s, 4H), 5.24-5.28 (m, 8H), 8.42-8.44 (m, 2H), 8.84-8.86 (m, 2H), 8.92-8.94 (m, 2H), 9.05-9.12 (m, 4H), 9.31-9.33 (m, 4H). LC-MS split peak >90% purity by peak area.

The following examples were prepared according to an analogous method to Example 4 from the corresponding pegylated starting material.

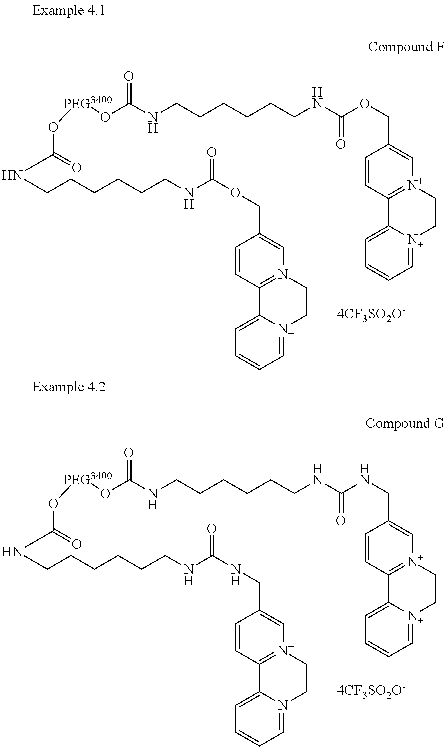

For Example 4.1, the pegylated starting material was prepared according to an analogous method to Preparation 6 using the di(6-isocyanatohexyl)carbamate prepared according to an analogous method to Preparation 7 using dihydroxy-PEG (3400)

### Example 4.1 NMR Data:

¹H NMR (DMSO-d, 500 MHz) δ 1.18-1.58 (m, 20H), 2.90-3.03 (m, 4H), 3.42-3.65 (m, ca. 308H), 5.23-5.27 (m, 8H), 5.42 (s, 4H), 8.48-8.50 (m, 2H), 8.82-8.84 (m, 2H), 8.94-8.96 (m, 2H), 9.10-9.12 (m, 4H), 9.35-9.37 (m, 4H).

For Example 4.2, the pegylated starting material was prepared according to an analogous method to Preparation 6 using 5-aminomethyl-2,2-bipyridyl and the di(6-isocyanatohexyl)carbamate prepared according to an analogous method to Preparation 7.

### Example 4.2

### NMR Data

¹H NMR (D₂O, 500 MHz) δ 1.19-1.62 (m, 20H), 2.90-3.12 (m, 4H), 3.50-3.92 (m, ca. 308H), 4.38-4.42 (m, 4H), 4.65-4.75 (m, 8H), 8.04-8.06 (m, 2H), 8.20-8.22 (m, 2H), 8.32-8.34 (m, 2H), 8.56-8.58 (m, 2H), 8.62-8.64 (m, 2H), 8.81-8.82 (m, 2H), 8.83-8.84 (m, 2H).

The compounds of the invention have good cytotoxic activity and may be tested in the following assays as exemplified in Examples 5, 6 and 7.

### Example 5

### In Vitro Cytotoxicity Study

### Materials and Methods

### Compounds.

Compounds A, B, C and D are respectively diquat ditriflate (6,7-dihydrodipyrido[1,2-a:2′,1′-c]pyrazine-5,8-diium bistrifluoromethanesulfonate; compound A) and compounds of Example 1 (compound B), Example 2 (compound C), and Example 3 (compound D) hereinabove respectively and Cisplatin was purchased from Sigma (Poole, Dorset). All were stored in the dark at 4° C. An accurately weighed amount of each compound was dissolved in DMSO and Hank's Balanced Salt Solution (Sigma) according to Table 2 below:

TABLE 2
Procedure for making 100 μM stock solutions

| | | Amount of | | =X mM | | | |
|---|---|---|---|---|---|---|---|
| Amount of | | | DMSO | HBSS | | 100 μM stock | |
| Com- | Mol. | Amount | added | added | (in | μl of | μl of |
| pound | Wt. | (mg) | (μl) | (μl) | 1 ml) | X | RPMI |
| A | 500 | 1 | 100 | 900 | 2.00 | 50 | 950 |
| B | 500 | 1 | 100 | 900 | 2.00 | 50 | 950 |
| C | 5000 | 10 | 200 | 800 | 2.00 | 50 | 950 |
| D | 5000 | 10 | 100 | 900 | 2.00 | 50 | 950 |
| Cisplatin | 300.05 | 1 | 100 | 900 | 3.33 | 30 | 970 |

Further dilutions were made in complete RPMI1640 cell culture medium (RPMI1640 cell culture medium supplemented with 10% foetal bovine serum, 1 mM L-glutamine, and 2 mM sodium pyruvate (all from Sigma)).

### Cell Lines.

H460 non-small cell lung adenocarcinoma, HT-29 colon adenocarcinoma and MCF-7 breast adenocarcinoma (ECACC, Salisbury, Wiltshire) were used for analysis. Cells were cultured in complete RPMI1640 cell culture medium, and maintained as monolayer cultures at 37° C. in a humidified 5% CO2 environment.

### MTT Assay.

Tumour cell growth inhibition was assessed using the MTT assay with a standard 96 hour plus an extended 10 day incubation. For the 96 hour assay 1×104 cells were inoculated into each well of a 96-well plate and incubated overnight at 37° C. in a humidified atmosphere containing 5% CO2 to give time to adhere to the wells and form a monolayer. For the 10 day incubation, 1×102 (H460 and HT-29) and 1×103 (MCF-7) cells were inoculated. The next day, culture medium was removed from each well and replaced with cell culture media for the untreated control, or media containing compound, and the plates then incubated for a further 96 hours or 10 days. A 5-log concentration range was used for each compound which was increased after the first run where no IC50 value (see below) was obtained. After 96 hours or 10 days culture medium was removed and 200 μL of 0.5 mgmL-1 MTT solution (Sigma) in complete medium added to each well. Following a further 4 hours incubation, the solution was removed from each well and 150 μL of DMSO added to solubilise the formazan crystals resulting from MTT conversion. Absorbance values for the resulting solutions were read at 550 nm on a Multiskan Ex microplate reader (Thermo Fisher, Loughborough, UK) and cell survival calculated as the absorbance of treated cells divided by the control. Results are expressed in terms of IC50 values (i.e. concentration of compound required to kill 50% of cells), which were determined manually in Microsoft Excel from plots of concentration versus percentage cell survival, and all experiments were performed in triplicate.

TABLE 3
in vitro cytotoxicity evaluation, 10 day exposure

| Cell | | | | | | |
|---|---|---|---|---|---|---|
| Line | Compound | Run 1 | Run 2 | Run 3 | Mean ^{a} | ±S.D. |
| H460 | A | 5.2 | 5.5 | 5.4 | 5.4 | 0.2 |
| | B | 4.7 | 4.7 | 5.0 | 4.8 | 0.2 |
| | C | >30 ^{b, c} | >100 ^{b} | >100 ^{b} | — ^{d} | — ^{d} |
| | D | >30 ^{b, c} | >100 ^{b} | >100 ^{b} | — ^{d} | — ^{d} |
| | Cisplatin | 1.1 | 1.0 | 0.9 | 1.0 | 0.1 |
| HT-29 | A | 1.8 | 0.9 | 1.7 | 1.5 | 0.5 |
| | B | 1.8 | 1.2 | 1.8 | 1.6 | 0.3 |
| | C | >30 ^{b, c} | 82 | 78 | — ^{d} | — ^{d} |
| | D | >30 ^{b, c} | >100 ^{b} | 81 | — ^{d} | — ^{d} |
| | Cisplatin | 2.4 | 1.3 | 0.7 | 1.5 | 0.9 |
| MCF-7 | A | 2.7 | 5.4 | 6.8 | 5.0 | 2.1 |
| | B | 2.7 | 2.3 | 5.9 | 3.6 | 2.0 |
| | C | >30 ^{b, c} | 97 | >100 ^{b} | — ^{d} | — ^{d} |
| | D | >30 ^{b, c} | 72 | >100 ^{b} | — ^{d} | — ^{d} |
| | Cisplatin | 2.3 | 2.3 | 2.5 | 2.4 | 0.1 |

Notes:
^{a} Results are IC₅₀ values in μM
^{b} No IC₅₀ value obtained for the concentration range used therefore
value not used in calculating means
^{c} For first run-through highest concentration used was 30 μM. As
no IC50 values were obtained, the highest concentration was increased
to 100 μM for the second and third run-throughs. For compounds C
and D, since there were problems with solubility, final DMSO concen-
tration had to be increased as follows, with 0.5% DMSO in medium (no
compound added) demonstrating toxicity in HT-29 cells (31% cell kill),
and 1% DMSO in medium (no compound added) demonstrating toxicity in
MCF-7 cells (29% cell kill), and HT-29 cells (39% cell kill):
DMSO concentrations

| | | | | | |
|---|---|---|---|---|---|
| | A | B | C | D | CIS |
| Run 1 | 0.15% | 0.15% | 0.30% | 0.15% | 0.09% |
| Run 2 | 0.15% | 0.15% | 1% | 0.50% | 0.09% |
| Run 3 | 0.15% | 0.15% | 1% | 0.50% | 0.09% |

^{d} Insufficient data to calculate mean ± S.D.

### Example 5a

### Combination Studies with Cisplatin

Compounds A and B were evaluated in combination with cis platin against four cell lines A2780 and A2780/cis [ovarian and cisplatin ovarian resistant lines], M 14 [melanoma] and MCF7 [Breast]. The compounds were tested over a 3-log dose range from 1 micromolar to 10 micromolar with respect to compounds A and B and 3 log dose range with respect to cisplatin at appropriate concentrations with regards the cell line under assay.

Results are provided in the Table 4 below.

TABLE 4
Combination with Cisplatin

| Cisplatin | Compound A μM | | | | Compound B μM | | | |
|---|---|---|---|---|---|---|---|---|
| μM | 0 | 1 | 3 | 10 | 0 | 1 | 3 | 10 |
| A2780 | | | | | | | | |
| 0 | 97 | 90.5 | 86.3 | 66 | 95.9 | 88.5 | 59 | 35.7 |
| 1 | 49.5 | 58 | 54.4 | 40.6 | 52.5 | 55.2 | 46.3 | 20.4 |
| 3 | 19.6 | 24.5 | 22.3 | 19 | 19.2 | 20.3 | 18.3 | 11.7 |
| 10 | 4.8 | 5.2 | 5.3 | 5.2 | 5.8 | 5.5 | 5.4 | 3.9 |
| A2780/cis | | | | | | | | |
| 0 | 87.8 | 77.8 | 48.7 | 29.4 | 87.6 | 74.4 | 44.7 | 15.4 |
| 10 | 36.3 | 51.4 | 31.9 | 16.6 | 39.7 | 32.1 | 24.2 | 9 |
| 30 | 8.7 | 10 | 10.2 | 6 | 8.6 | 6.5 | 8.1 | 5 |
| 100 | −0.5 | −1.6 | 1.9 | 1.8 | 1.1 | 0 | 3.3 | 2.1 |
| M14 | | | | | | | | |
| 0 | 86.3 | 87 | 80 | 56.5 | 95.6 | 93.2 | 79.7 | 59.2 |
| 1 | 85.6 | 83.1 | 75.8 | 49 | 97.7 | 88.4 | 78.9 | 55.7 |
| 3 | 77.1 | 71.3 | 68 | 44.4 | 84.8 | 83.8 | 72 | 50.2 |
| 10 | 58.3 | 57.9 | 55.4 | 36.4 | 63.3 | 58.4 | 47.1 | 33.4 |
| MCF-7 | | | | | | | | |
| 0 | 111.7 | 109 | 95.1 | 52.6 | 90.1 | 76.2 | 69.1 | 67.3 |
| 0.1 | 107.4 | 96.4 | 80.9 | 52.8 | 110.8 | 87.3 | 81.4 | 63.7 |
| 0.3 | 76 | 69.4 | 64.8 | 39.6 | 91.3 | 77.9 | 73.2 | 51.7 |
| 1 | 72.4 | 62 | 66.2 | 32.6 | 70.3 | 72.3 | 74.3 | 46.3 |

Notes:
^{a}Results are % cell survival

### Example 5b

### Cell Death in HEK293 Cells after 48 Hours Exposure

The ability of test compounds to impact cell death in HEK293 cells after 48 hours exposure was assessed.

### Method

### Cell Death Assay

HEK293 cells were seeded into a 24 well plate (500×105 cells/well) in DMEM supplemented with 10% FCS, antibiotics and L-glutamine. Drugs at indicated concentrations were added followed by incubation for 48 hours at 37° C. before HEK293 cells were washed, trypsinised and resuspended in PBS prior to PI staining (0.1 μg/ml) before analysis of PI+ ve cells by flow cytometry.

Example 4.1 (Compound F) was tested in the above assay.

### Results

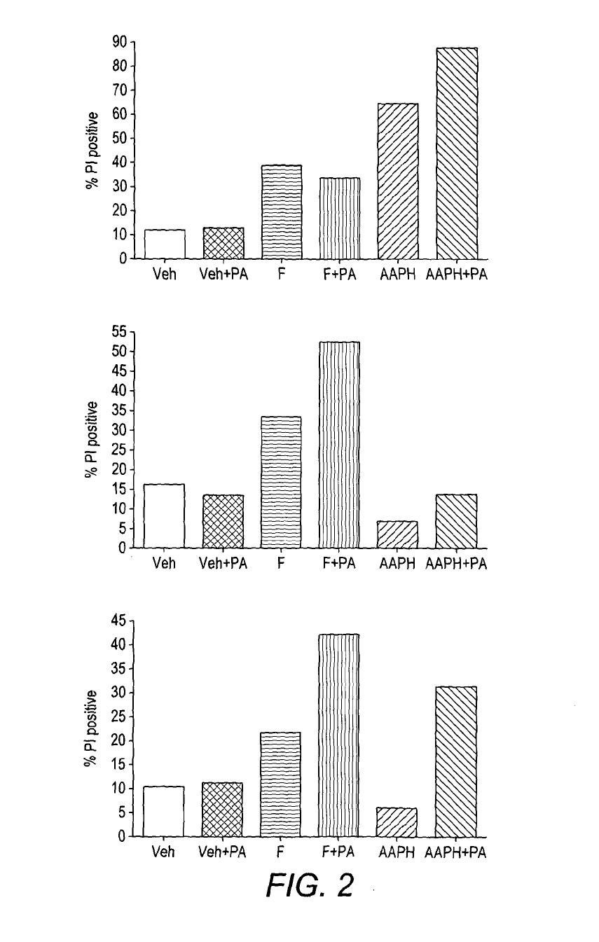

FIG. 2 shows the ability of compound F (100 μM) and the positive control used to induce ROS production, AAPH (5 mM), to induce cell death (PI staining of cells) in HEK293 cells above levels associated with vehicle treatment (Veh) in the absence and presence of pluronic acid (0.1%; PA). Data are presented from three independent experiments. Compound F (Example 4.1, 100 μM) induced cell death in HEK293 cells above vehicle treatment (FIG. 2), which was facilitated by the presence of pluronic acid (0.1%) in 2/3 experiments. The positive control to induce ROS production, AAPH (5 mM) inducedcell death in HEK293 cells in 2/3 experiments in the presence of pluronic acid (0.1%).

### Example 6

### Studies of Generation of Reactive Oxygen Species and Cell Death

### i) ROS Production in Isolated Mitochondria

The ability of test compounds to impact ROS production in isolated mitochondria after 120 mins exposure was assessed.

### Materials and Methods

### Compounds.

Compounds A, B, F and G are respectively diquat ditriflate (6,7-dihydrodipyrido[1,2-a:2′,1′-c]pyrazine-5,8-diium bistrifluoromethanesulfonate; compound A) and compounds of Example 1 (compound B), Example 4.1 (compound F), and Example 3 (compound G) hereinabove respectively

### Isolation of Mitochondria and ROS Assay

Mitochondria were isolated from harvested human embryonic kidney (HEK293) cells (20×10⁶) using a commercial kit (Thermo Scientific) before re-suspension in PBS to a final volume of 800 μL. The mitochondria were then incubated with DCFDA (10 μM, 30 minutes, room temperature) before incubation with drug (Compounds A, B, G and F [100 μM] or AAPH [5.0 mM]) for 120 mins at room temperature with ROS production detected using a FluoroSkan. Independent experiments (each assessing the impact of all four compounds along with a positive [AAPH; 5.0 mM] and vehicle control) were repeated three times.

### Results

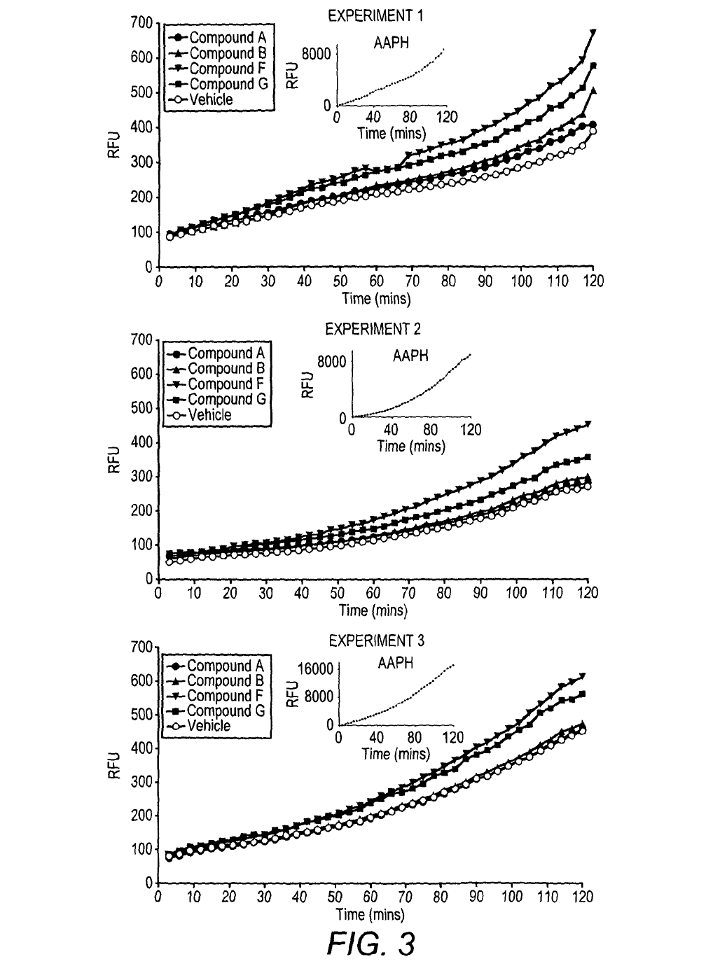

FIG. 3 shows the ability of compounds A, B, F and G (each at 100 μM) or the positive control, AAPH (5.0 mM; inset), to impact ROS production from human mitochondria isolated from HEK293 cells. Data are presented from three independent experiments. Compounds F and G (each at 100 μM) robustly increased ROS production from human isolated mitochondria above vehicle treatment (FIG. 3). Compound B (100 μM) induced a more modest increase, whilst ROS production in the presence of Compound A (100 μM) was barely above vehicle levels. The rank order of efficacy was F>G>B>A.

### Ii) ROS Production in Intact HEK293 Cells

The ability of test compounds to impact ROS production in intact HEK293 cells after 48 hours exposure was assessed.

### Method

### ROS Assay

HEK293 cells were stained with 2′,7′-dichlorofluorescein diacetate (DCFDA; 10 μM, 30 minutes, 37° C.). The cells were then washed and resuspended to 250×105/ml. The stained HEK293 cells were seeded into a 24 well plate (500×105 cells/well) in DMEM supplemented with 10% FCS, antibiotics and L-glutamine. Drugs at indicated concentrations were added followed by incubation for 48 hours at 37° C. before HEK293 cells were washed, trypsinised and resuspended in PBS prior to analysis for ROS production by flow cytometry.

Example 4.1 (Compound F) was tested in the above assay.

### Results

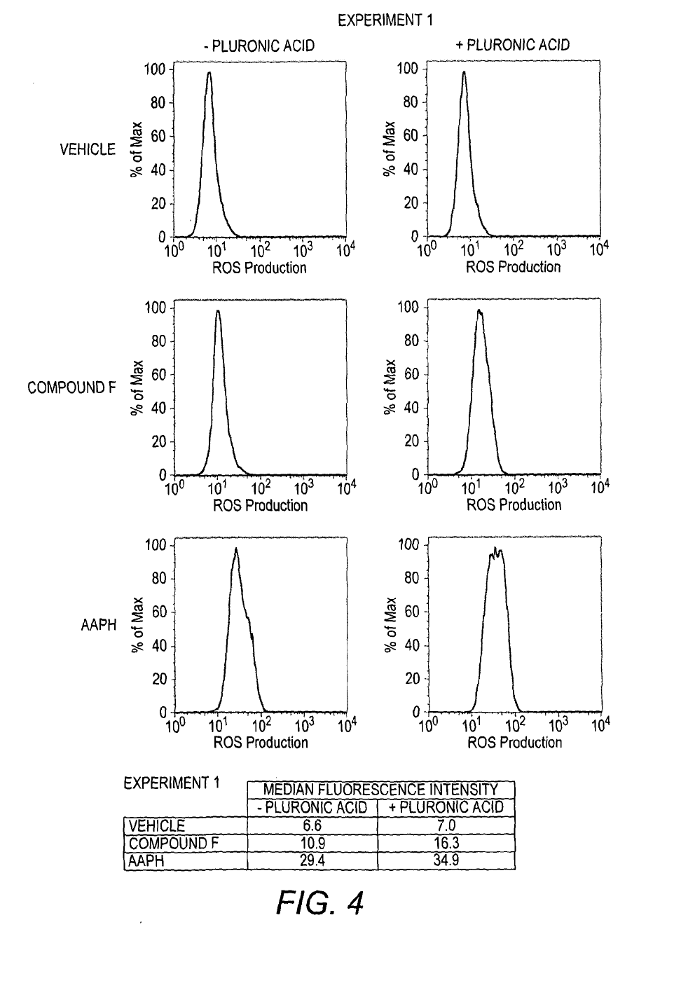

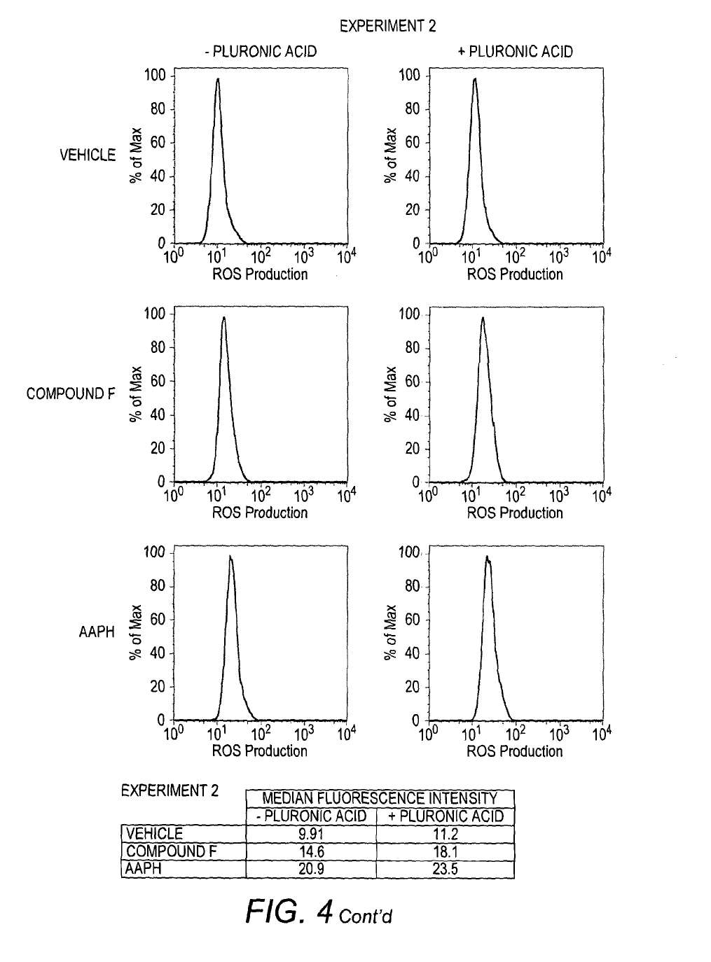

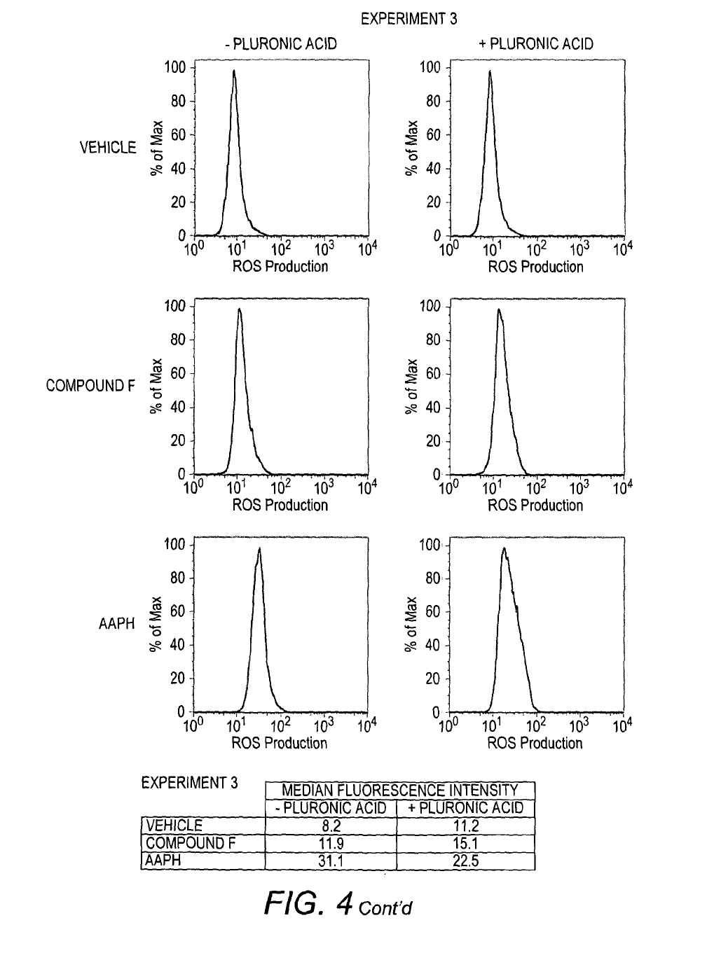

FIG. 4 shows the ability of compound F and the positive control, AAPH (5.0 mM), to promote ROS production in HEK293 cells above vehicle treatment in the absence and presence of pluronic acid (0.1%). Data are presented from three independent experiments. Compound F (100 μM) increased ROS production in HEK293 cells above vehicle treatment (See FIG. 4) which tended to be facilitated by the presence of pluronic acid, a mild surfactant. (0.1%). The positive control, AAPH (5 mM) induced ROS production in the absence and presence of pluronic acid (0.1%). Compound F displays the ability to increase ROS production and promote cell death in HEK293 cells. There is a trend (2/3 experiments) for the non-ionic surfactant, pluronic acid to increase the cellucidal action of Compound F and a consistent but small increase in the ability to increase ROS production relative to vehicle treatment, suggesting an improvement of intracellular delivery of Compound F.

### Example 7

### Xenograft Model Study

### Evaluation of the Efficacy of the Compounds C & D (Examples 2 & 3 Respectively) in the MCF-7 Human Breastadenocarclnoma Xenograft Model

TABLE 5
Dosing Regimen for Xenograft Studies

| | Growth | | Maximum |
|---|---|---|---|
| | delay | | % weight |
| Treatment | (days) | Significance | loss |
| Saline controls, i.v., d 0, 2, 4, 7, 9 & | | — | 1.0 |
| 11 | | | |
| Compound C (Example 2), 200 mg/ | 3.1 | p > 0.05 ns | 0 |
| kg, i.v., d 0, 2, 4, 7, 9 & 11 | | | |
| Compound D (Example 3), 200 mg/ | 2.4 | p > 0.05 ns | 0 |
| kg, i.v., d 0, 2, 4, 7, 9 & 11 | | | |
| Cisplatin, 10 mg/kg, i.p., d 0 | 5.1 | p < 0.01 | 8.0 |

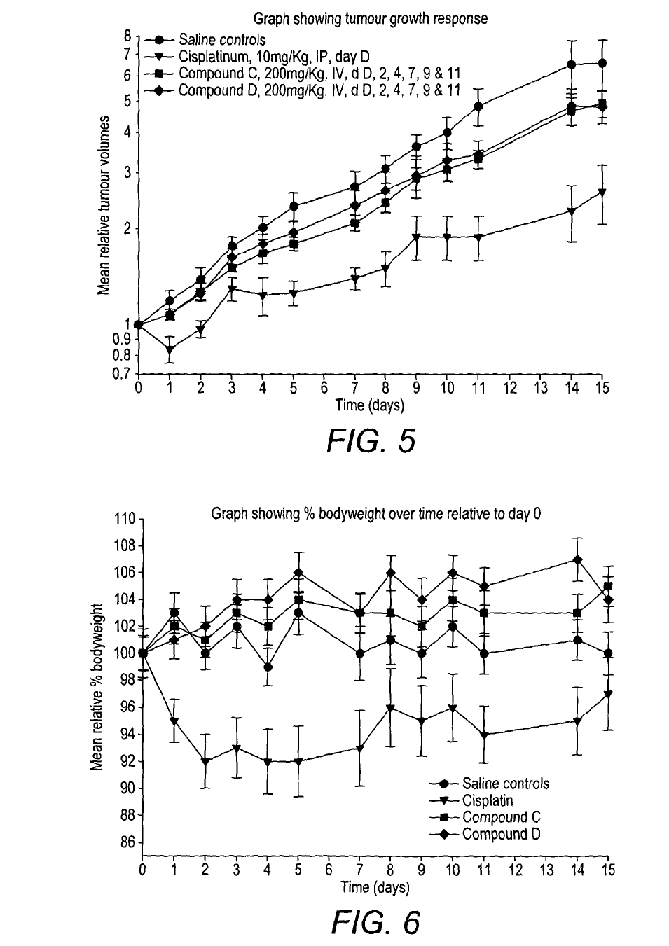

FIG. 5 shows the tumor growth response to the dosing regimen outlined in Table 5,

FIG. 6 shows the % bodyweight change during the dosing regimen relative to day 0.

In conclusion, a significant retardation of tumour growth was observed in mice to which test compound had been administered intraveneously, when compared to saline controls. This was observed without evidence of macro toxicity.

### Example 8

### Redox Potential Study

### Experimental Set-Up

### Instrument:

BioAnalytical Systems CV50W potentiostat (from BAS Inc., USA)

### Technique:

Square wave voltammetry (4 mV step, 25 mV pulse, 10 Hz SW frequency)

### Electrodes:

3 mm diameter glassy carbon working electrode (from BAS)

3 mm diameter glassy carbon counter electrode (from BAS)

Saturated calomel reference electrode (SCE, from Sycopel Scientific Ltd., UK)

### Electrolyte:

0.1 mol dm-3 lithium bis(trifluoromethanesulphonyl)imide ((LiNTf2)CAS 90076-65-6, from 3M)

### Measurement Temperature:

20° C.

Voltammograms were recorded from 0.5 V to −1.2 V in deoxygenated solutions of the Compounds.

### Samples:

Compounds A, B, C and D are respectively diquat ditriflate (A) and compounds of Examples 1, 2, and 3 respectively

| Sample | Wt sample/mg | Vol. electrolyte/cm³ |
|---|---|---|
| A | 12.0 | 25.0 |
| B | 15.0 | 25.0 |
| C | 8.5 | 25.0 |
| D | 19.5 | 25.0 |

### Results:

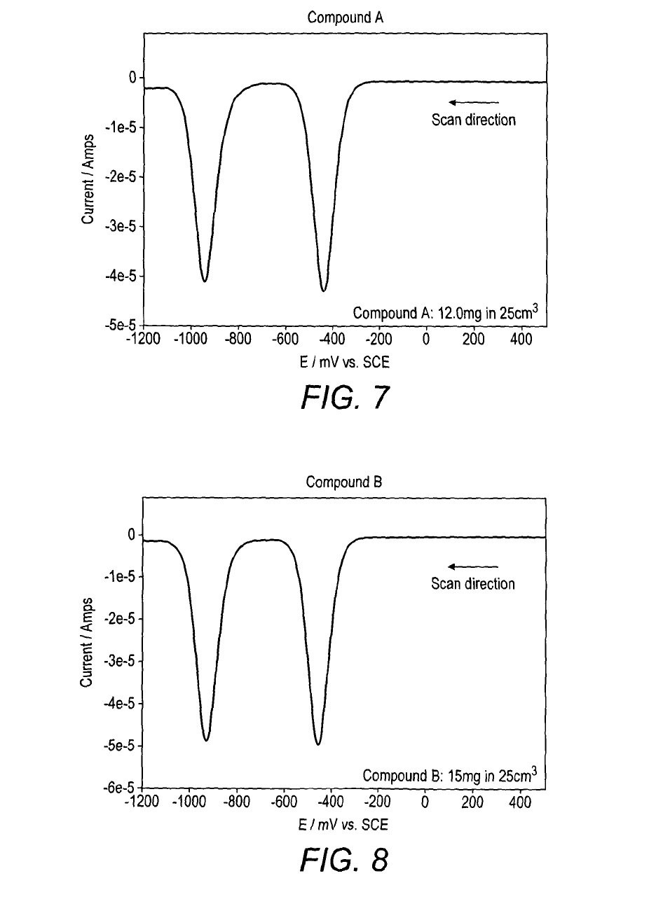

Compound A: FIG. 7 displays the SWV for Compound A

Reduction potential 1=−0.444 V vs. SCE

Reduction potential 2=−0.944 V vs. SCE

These values were highly reproducible at t lmV

Compound B: FIG. 8 displays the SWV for Compound B

Reduction potential 1=−0.460 V vs. SCE

Reduction potential 2=−0.932 V vs. SCE

These values were highly reproducible at ±1 mV

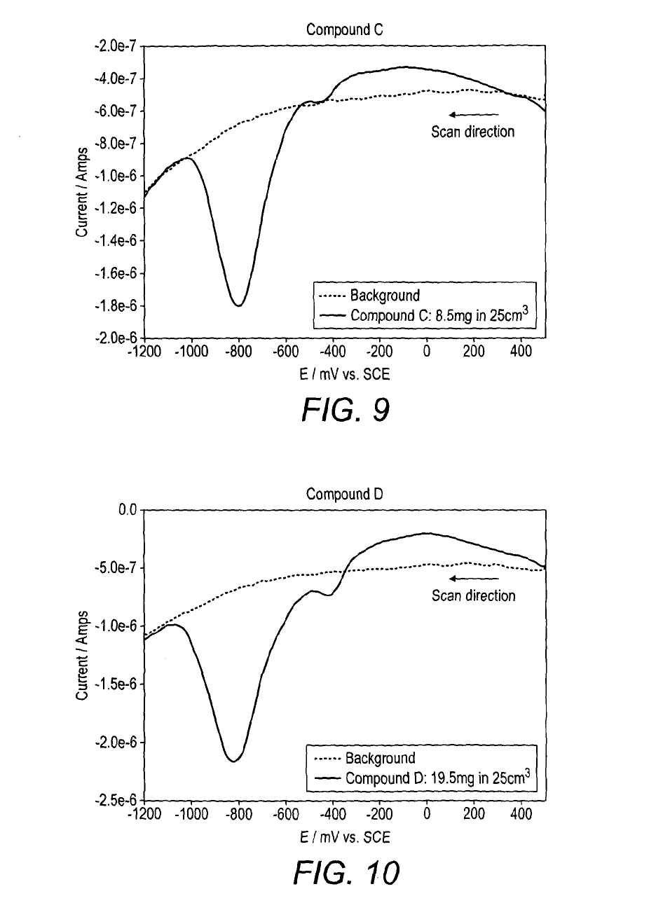

Compound C: FIG. 9 displays the SWV for 8.5 mg of Compound C in 0.1 mol dm⁻³ LiNTf. (solid line) and a background voltammogram (dotted line).

For compound C two reductions were observed, one very small process at −0.470 V vs. SCE (first scan) and a large main reduction at −0.803 V vs. SCE (first scan). Repetitive voltammetric cycling reduced the main peak potential to −0.876 mV. Inspection of the electrode's surface indicated significant adsorption of the compounds on the electrode following reduction (which relocates reduction potentials) so only the first scan is accurate. The values for the first scan are as follows;

Reduction potential 1=−0.470 V vs. SCE

Reduction potential 2=−0.803 V vs. SCE

“First scan” means the first scan recorded following polishing of the electrode to provide a clean surface.

### Note:

The first reduction at −0.470 V vs. SCE could be an artifact due to adsorption.

No further reduction processes were observed down to −1.5 V vs. SCE.

Compound D: FIG. 10 displays the SWV for 19.5 mg of Compound D in 0.1 mol dm-³ LiNTf (solid line) and a background voltammogram (dotted line).

Again, for compound D two reductions were observed, one very small process at −0.423 V vs. SCE and a large main reduction at −0.828 V vs. SCE. Repetitive voltammetriccycling reduced the main peak potential to −0.901 mV. Inspection of the electrode indicated significant adsorption of the compounds on the electrode following reduction (which relocates reduction potentials) so only the first scan is accurate. The values for the first scan are as follows;

Reduction potential 1=−0.423 V vs. SCE

Reduction potential 2=−0.828 V vs. SCE

### Note:

The first reduction at −0.423 V vs. SCE could be an artifact due to adsorption.

No further reduction processes were observed down to −1.5 V vs. SCE.

In conclusion, the compounds tested exhibit biologically relevant reduction potentials evidencing their potential to decouple the electron flow in normal biological reduction pathways by abstracting one electron and transferring it to molecular oxygen [O₂], forming superoxide [O₂⁻], in the process.

### Preparation 1

### 5-hydroxymethyl-2,2-bipyridyl

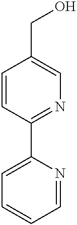 [SMILES: OCc1ccc(-c2ccccn2)nc1]

To a solution of 5-methyl carboxylate-2,2-bipyridyl (Preparation 3, 1.00 g, 4.67 mmol) in anhydrous THF (100 mL) cooled to −78° C. was added lithium aluminium hydride (0.18 g, 4.67 mmol) portion-wise. The mixture was stirred at −78° C. for 30 minutes then allowed to warm to −20° C. whereupon the mixture became homogeneous. After 1 hour at −20° C. TLC analysis indicated reaction completion (CH₂Cl₂: MeOH) (90:10). The mixture was cooled to −78° C. once more and carefully quenched with 10% aqueous THF (15 mL). The mixture was allowed to warm to room temperature, stirred with Celite for 15 minutes and then filtered and washed through with CH₂Cl₂. The filtrate was washed with brine (100 mL). The organic layer was dried over MgSO₄, filtered and concentrated in vacuo to afford the title compound as a red oil which slowly solidified (830 mg, 95% yield).

¹H NMR (CDCl₃, 500 MHz) δ 1.96 (bs, 1H), 4.72 (d, 2H), 7.24 (m, 1H), 7.76 (m, 2H), 8.31 (d, 2H), 8.61 (m, 2H);

MS-ES⁺ m/z 187.3, [M+H]⁺.

### Preparation 2

### Ethane Ditriflate (DTE)

### 2-(trifluoromethylsulfonyloxy)ethyl trifluoromethanesulfonate

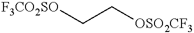

A mixture of ethylene glycol (1.80 mL, 32.22 mmol) and anhydrous pyridine (5.21 mL, 64.44 mmol) in anhydrous dichloromethane (20 mL) was added drop-wise over 20 minutes to an ice-cooled solution of trifluoromethane sulfonic anhydride (10.60 mL, 64.44 mmol) in anhydrous dichloromethane (40 mL). The resultant red suspension was stirred in an ice bath under nitrogen for an additional 10 minutes then washed with H₂O (2×60 mL). The organic layer was dried over MgSO₄, filtered and concentrated in vacuo @ 30° C. to yield the title compound as a red oil which solidified on storage under nitrogen @<−10° C. (9.71 g, 92% yield).

¹H NMR (CDCl₃, 500 MHz) δ 4.68 (s, 4H).

### Preparation 3

### 5-methyl carboxylate-2,2-bipyridyl

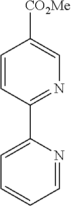 [SMILES: COC(=O)c1ccc(-c2ccccn2)nc1]

To a solution of 5-carboxylic acid-2,2-bipyridyl (Preparation 4, 1.60 g, 5.88 mmol) in methanol (32 mL) was added conc. HCl (2 drops, catalytic) and the mixture was refluxed overnight under nitrogen.

The solution was concentrated in vacuo and then diluted with a mixture of 10% aqueous Na₂CO₃ solution (50 mL) and ethyl acetate (50 mL). The aqueous phase was separated and extracted with ethyl acetate (1×50 mL). The combined organics were dried over MgSO₄, filtered and concentrated in vacuo to afford the title compound as a white solid (780 mg, 61% yield).

¹H NMR (CDCl₃, 500 MHz) δ 3.99 (s, 3H), 7.36 (m, 1H), 7.86 (m, 1H), 8.41 (dd, 1H), 8.50 (m, 2H), 8.72 (m, 1H), 9.28 (d, 1H);

MS-ES⁺ m/z 214.2, [M+H]⁺.

### Preparation 4

### 5-carboxylic acid-2,2-bipyridyl

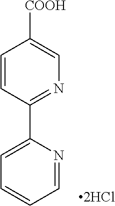 [SMILES: O=C(O)c1ccc(-c2ccccn2)nc1]

A mixture of 5-methyl-2,2-bipyridyl (1.00 g, 5.88 mmol), potassium permanganate (3.34 g, 21.14 mmol) and water (40 mL) was heated to reflux and stirred overnight under nitrogen.

The reaction mixture was allowed to cool to room temperature and filtered through Celite. The Celite cake was washed with water (2×10 mL) and the combined filtrates were acidified to pH 1 with conc. HCl. The solution was concentrated to dryness in vacuo to afford an off-white solid, which was triturated in acetone to furnish the title compound as a white solid (1.60 g, quantitative yield).

¹H NMR (DMSO-d₆, 500 MHz) δ 4.49 (bs, 1H), 7.61 (m, 1H), 8.12 (m, 1H), 8.44 (dd, 1H), 8.53 (m, 1H), 8.57 (m, 1H), 8.77 (dd, 1H), 9.19 (s, 1H);

MS-ES⁺ m/z 201.2, [M+H]⁺

### Preparation 5

### 5-methyl-2,2-bipyridyl mPEG (5000) ether

### (5-((2-(2-methoxyethoxy)(ethoxy)ₙ)methyl)-2,2′-bipyridine)

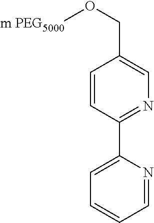

To a suspension of NaH (48 mg, 1.18 mmol) in anhydrous THF (8 mL) was added a solution of 5-hydroxymethyl-2,2-bipyridyl (Preparation 1, 220 mg, 1.18 mmol) in anhydrous THF (12 mL) drop-wise. The mixture was stirred for 20 minutes at room temperature and a warm solution of mPEG (5000) mesylate (4.00 g, 0.79 mmol) in anhydrous THF (20 mL) was added drop-wise.

The red solution was heated at reflux temperature overnight. The mixture was allowed to cool to room temperature and filtered through Celite, washing through with CH₂Cl₂. The filtrate was concentrated in vacuo to ca. 2 mL volume and slurried in ether (60 mL).

The resultant precipitate was collected by filtration to afford the title compound as a pale pink solid (2.72 g, 67% yield).

¹H NMR (CDCl₃, 500 MHz) δ 3.37 (s, 3H), 3.64 (m, ca. 452H), 4.65 (s, 2H), 7.32 (m, 1H), 7.82 (m, 2H), 8.37 (m, 2H), 8.62 (m, 1H), 8.65 (m, 1H). UV-VIS λₘₐₓ 280 nm.

### Preparation 6 [2,2′-bipyridin]-5-ylmethyl (2-(2-(2-methoxyethoxy)(ethoxy)ₙ)ethyl) hexane-1,6-diyldicarbamate

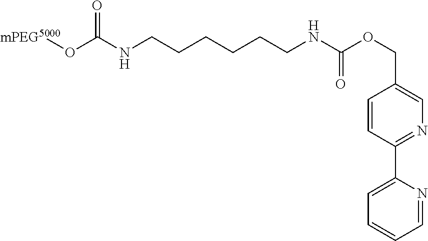

To a mixture of mPEG (5000)-isocyanate (Preparation 7, 5.55 g, 1.07 mmol) in anhydrous chloroform (75 mL) was added 5-hydroxymethyl-2,2-bipyridyl (0.50 g, 2.68 mmol) followed by dibutyltin dilaurate (0.10 mL, catalytic). The solution was stirred at room temperature under nitrogen overnight.

The mixture was concentrated in vacuo to ca. 5 mL volume and the residue was slurried in ether (150 mL). The solid was collected by filtration and dissolved in the minimum quantity of CH₂Cl₂. Ether (150 mL) was added and the precipitate was again collected by filtration to afford the title compound as an off-white solid (4.85, 85% yield).

¹H NMR (CDCl₃, 500 MHz) δ 1.22-1.49 (m, 12H), 3.38 (s, 3H), 3.64 (m, ca. 452H), 4.12 (bs, 1H), 4.90 (bs, 1H), 5.16 (s, 2H), 7.32 (m, 1H), 7.84 (m, 2H), 8.38 (m, 2H), 8.66 (m, 2H).

UV-VIS λₘₐₓ 285 nm.

### Preparation 7

### 2-(2-methoxyethoxy(ethoxy)ₙ)ethyl (6-isocyanatohexyl)carbamate

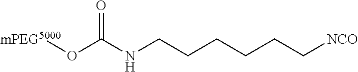

A suspension of mPEG (5000) —OH (10.00 g, 2.00 mmol) in anhydrous toluene (125 mL) was azeodried at reflux for 3 hours via a Dean-Stark apparatus.

The solution was allowed to cool to room temperature and added rapidly drop-wise, under nitrogen, to a mixture of 1,6-diisocyanatohexane (1.60 mL, 10.00 mmol) and dibutyltin dilaurate (0.04 mL, 0.07 mmol). Upon addition, the reaction was subsequently stirred at room temperature under nitrogen overnight.

Anhydrous hexane (200 mL) was added to the reaction solution rapidly drop-wise with stirring and the resultant precipitate was collected by filtration under nitrogen and washed with anhydrous hexane (30 mL). The isolated solid was slurried in anhydrous toluene (50 mL) and re-precipitated with anhydrous hexane (100 mL). The solid was collected by filtration, washed with anhydrous hexane (30 mL) and dried under nitrogen for 15 minutes to afford the title compound as a white solid (9.91 g, 96% yield). The material was used immediately in the subsequent reaction and stored under nitrogen.

### Preparation 8

### 1,2-bis-(5-ylmethoxy)(ethoxy)_{n[}2,2′-bipyridine]

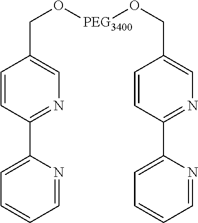

To a solution of 5-hydroxymethyl-2,2-bipyridyl (Preparation 1, 415 mg, 2.23 mmol) in anhydrous THF (40 mL) was added sodium hydride (92 mg, 2.23 mmol). The mixture was stirred at room temperature for 15 mins and a solution of PEG (3400) dimesylate (2.00 g, 0.56 mmol) in anhydrous THF (40 mL) was added drop-wise. The mixture was stirred for 12 hours under nitrogen at 80° C.

The mixture was allowed to cool to room temperature and concentrated in vacuo. The residue was dissolved in CH₂Cl₂ (80 mL) and washed with brine (2×50 mL). The organic layer was dried over MgSO₄, filtered and diethyl ether (180 mL) was added drop-wise at room temperature. The resultant precipitate was collected by filtration, washed with diethyl ether (20 mL) and dried to afford the title compound as a cream coloured solid (1.79 g, 85% yield). ¹H NMR (DMSO-d, 500 MHz) δ 3.49-3.65 (m, ca. 308H), 4.62 (s, 4H), 7.42-7.46 (m, 2H), 7.85-7.99 (m, 4H), 8.38-8.42 (m, 4H), 8.64-8.66 (d, 2H), 8.68-8.71 (m, 2H). LC-MS>90% purity by peak area.

1. A compound of Formula Ih: 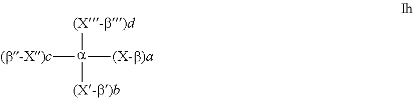
or a pharmaceutically acceptable salt or solvate thereof, wherein
a, b, c, d are independently 0 or 1 wherein at least one of a, b, c and d is 1;
α is polyethylene glycolyl or H, wherein when α is H or a monofunctional polyethylene glycolyl, α is 1, b is 0, c is 0 and d is 0;
X, X′, X″ and X′″ are each independently a linker group wherein the linker group is selected from —O—, —C(O)NR¹⁰—, —NR⁰C(O)NR¹¹—, —NR⁰C(O)O—, —NR¹⁰—, —C(O)O—, —S—, —(SO₂)NR¹⁰—, —(SO₂)O—, —NR¹⁰(SO₂)O—, —NR¹⁰(SO₂)NR¹¹—, —NR¹⁰C(O)NR¹¹(CH₂)ₙNR^{10a}C(O)O—, —OC(O)NR⁰(CH₂)ₙNR^{10a}C(O)NR^{11a}—, —NR¹⁰C(O)NR¹ (CH₂)ₙNR^{10a}C(O)NR^{11a}—, and —OC(O)NR¹⁰(CH₂)ₙNR^{10a}C(O)O—, wherein each (CH₂) is optionally substituted by one or more halogen atoms, hydroxyl, C₁-C₄ alkoxy, C(O)NH₂, C(O)NHC₁-C₆ alkyl or C(O)N(C₁-C₆ alkyl)₂;
R¹⁰, R^{10a}, R¹ and R^{11a} are independently selected in each occurrence from H, C₁-C₈ alkyl; C₃-C₈ cycloalkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; wherein the alkyl groups are optionally substituted by one or more halogen atoms, hydroxyl, C₁-C₄ alkoxy, C(O)NH₂, C(O)NHC₁-C₆ alkyl or C(O)N(C₁-C₆ alkyl)₂;
n is 1, 2, 3, 4, 5 or 6;
β, β′, β″ and β′″ are each independently a dipyridinium salt wherein the dipyridinium salt is of Formula 2 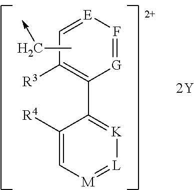
wherein E, F, G, K, L and M are each independently selected from CR¹ and NR² with the proviso that E, F or G is NR² and K, L or M is NR² and only one of E, F and G is NR² and only one of K, L and M is NR²; any pyridyl carbon atom may be the site of substitution of the methylene group bonded to the X linker;
R¹ and R² are independently selected from H and C₁₋₃ alkyl; or
wherein G and K are both NR², the two R² groups may be joined to form a CR¹²R¹³CR¹⁴R¹⁵ bridge;
R³ and R⁴ are independently selected from H and C₁₋₃ alkyl; or
R³ and R⁴ are joined to form a CR¹⁶CR¹⁷ bridge
R¹², R¹³, R¹⁴ and R¹⁵ are independently selected from H, C₁₋₈ alkyl; C₃-C₈ cycloalkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; wherein the alkyl groups are optionally substituted by one or more halogen atoms, hydroxyl, C₁-C₄ alkoxy, C(O)NH₂, C(O)NHC₁-C₆ alkyl or C(O)N(C₁-C₆ alkyl)₂;
R¹⁶ and R¹⁷ are independently selected from H, C₁-C₈ alkyl; C₃-C₈ cycloalkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroaioms selected from N, O and S, optionally substituted by one or more groups selected from halogen, oxo, C₁-C₆ alkyl and C(O)C₁-C₆ alkyl; wherein the alkyl groups are optionally substituted by one or more halogen atoms, hydroxyl, C₁-C₄ alkoxy, C(O)NH₂, C(O)NHC₁-C₆ alkyl or C(O)N(C₁-C₆ alkyl)₂;
Y⁻ is independently a pharmaceutically acceptable counteranion of an inorganic or organic acid; and
the arrow head denotes the point of attachment to X.

2. A compound according to claim 1 of Formula Ig:
β′-X′-α-X-β Ig
or a pharmaceutically acceptable salt or solvate thereof wherein α is a bifunctional polyethylene glycolyl group, and X, X′, β and β′ are as defined in claim 1.

3. A compound according to claim 1 of Formula I:
σ-X-β (I)
or a pharmaceutically salt or solvate thereof, wherein α is polyethylene glycolyl or H; and X and β are as defined in claim 1

4. A compound according to claim 1, wherein X, X′, X″ and X′″ are identical when present.

5. A compound according to claim 1, wherein β, β′, β″ and β′″ are identical when present.

6. A compound according to claim 1, wherein α is polyethylene glycolyl of molecular weight 100 to 20,000 daltons.

7. A compound according to claim 1, wherein α is methoxy polyethylene glycolyl.

8. A compound according to claim 1, wherein R¹ is H.

9. A compound according to claim 1, wherein X is selected from —O—, —C(O)NR¹⁰—, —NR¹⁰C(O)NR¹¹—, —NR¹⁰C(O)O—, —NR¹⁰—, —C(O)O—, —NR¹⁰C(O)NR¹¹(CH₂)ₙNR^{10a}C(O)O—, —OC(O)NR⁰(CH₂)ₙNR^{0a}C(O)NR^{11a}—, —NR¹⁰C(O)NR¹¹(CH₂)ₙNR^{10a}C(O)NR^{11a}—, and —OC(O)NR¹⁰(CH₂)ₙNR^{10a}C(O)O—.

10. A compound according to claim 1, wherein R¹⁰, R^{10a}, R¹¹ and R^{11a} are H.

11. A compound according to claim 1, wherein R³ and R⁴ are H.

12. A compound according to claim 1, wherein G and K are both NR², and the two R² groups are joined to form a C₂H₄ bridge

13. A compound according to claim 1, wherein β, β′, β″ and β′″ are each independently a dipyridinium salt of Formula 2b 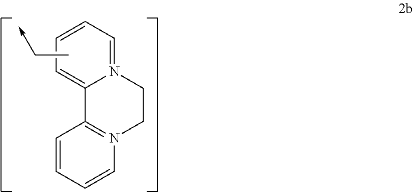
wherein Y⁻ is as defined in claim 1.

14. A compound according to claim 1, which is a compound of Formula 1a 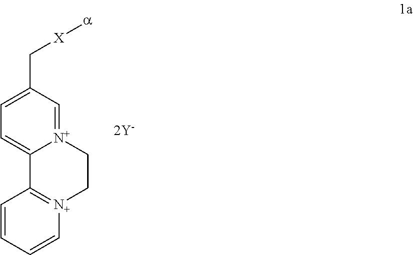
or a pharmaceutically acceptable salt or solvate thereof wherein α, X and Y⁻ are as defined in claim 1.

15. A compound according to claim 1, which is a compound of Formula Ij 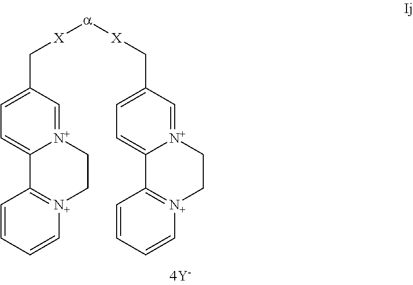
or a pharmaceutically acceptable salt or solvate thereof wherein G, X and Y⁻ are as defined in claim 1.

16. A compound according to claim 1, wherein R³ and R⁴ are joined to form a C₂H₂ bridge

17. A compound according to claim 1, wherein β, β′, β″ and β′″ are each independently a dipyridinium salt of Formula 2d 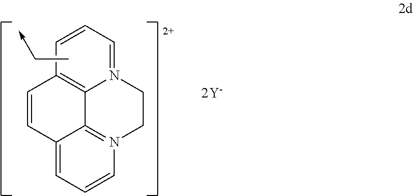
wherein Y⁻ is as defined in claim 1.

18. A compound according to claim 1, wherein Y⁻ is trifluoromethylsulfonate.

19. A compound according to claim 1 selected from:
3-(hydroxymethyl)-6,7-dihydrodipyrido[1,2-a:2′,1′-c]pyrazine-5,8-diium bistrifluoromethanesulfonate 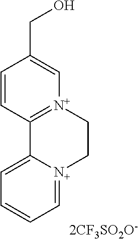 [SMILES: OCc1ccc2[n+](c1)CC[n+]1ccccc1-2]
3-((2-(2-methoxy-ethoxy)(ethoxy)ₙ)methyl)-6,7-dihydrodipyrido[1,2-a:2′,1′-c]pyrazine-5,8-diium bistrifluoromethanesulfonate. 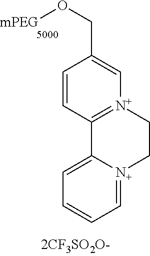
3-(((2-(2-methoxyethoxy)(ethoxy)ₙ)ethyl) hexane-1,6-diyldicarbamate)methyl)-6,7-dihydrodipyrido[1,2-a:2′,1′-c]pyrazine-5,8-diium bistrifluoromethanesulfonate 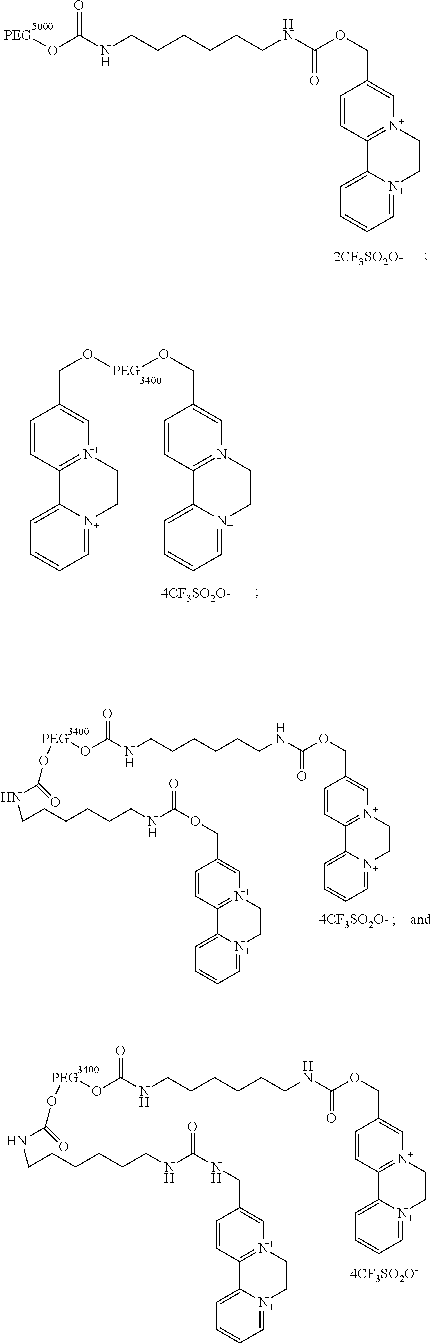
or a pharmaceutically acceptable salt or solvate thereof.

20. A pharmaceutical composition including a compound according to claim 1 and one or more pharmaceutically acceptable excipients, diluents and/or carriers.

21. A pharmaceutical composition according to claim 20 in combination with one or more other therapeutic agents.

22. A pharmaceutical composition according to claim 20 in combination with a photochemotherapy agent.

23. A compound according to claim 1 for use as a pharmaceutical.

24. A compound according to claim 23 for use in treating or preventing a disease or disorder characterised by pathologically proliferating cells.

25. Use of a compound according to claim 1 in the manufacture of a medicament for the prevention or treatment of a disease or disorder characterised by pathologically proliferating cells.

26. A method for preventing or treating a disease or disorder characterised by pathologically proliferating cells in which an effective amount of a compound according to claim 1 is administered to a patient in need of such treatment.

27. A process for preparing a compound according to claim 1 or a pharmaceutically acceptable salt or solvate thereof comprising the step of:
a) wherein α is polyethylene glycolyl, quaternisation of a compound of formula II 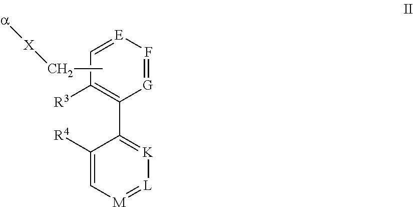
by reacting with a compound of formula V (R²—Y) under conventional quaternisation conditions wherein X, Y, R³ and R⁴ are as defined in claim 1; E, F, G, K, L and M are each independently selected from CR¹ and N with the proviso that E, F or G is N and K, L or M is N and only one of E, F and G is N and only one of K, L and M is N; R¹ and R² are as defined in respect of a compound of formula I; or
b) wherein a is H, quaternisation of a compound of formula VI 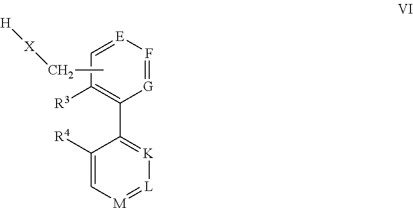
by reacting with a compound of formula V (R²—Y) under conventional quaternisation conditions,
wherein X, Y, R³ and R⁴ are as defined in claim 1; E, F, G, K, L and M are each independently selected from CR¹ and N with the proviso that E, F or G is N and K, L or M is N and only one of E, F and G is N and only one of K, L and M is N; and R¹ and R² are as defined in respect of a compound of formula I.
